(19) United States
(12) Patent Application Publication
(10) Pub. No.: US 2023/0201617 A1
(43) Pub. Date: Jun. 29, 2023

(54) Method and device that life energy spectrum derived from natural world has an important influence on humans and communication between humans and all things in nature

(71) Applicants: Lin ZHOU, Towaco, NJ (US); Sua xueShan ZHANG, Towaco, NJ (US); Fan ZHOU, Towaco, NJ (US)

(72) Inventors: Lin ZHOU, Towaco, NJ (US); Sua xueShan ZHANG, Towaco, NJ (US); Fan ZHOU, Towaco, NJ (US)

(21) Appl. No.: 17/300,955

(22) Filed: Dec. 27, 2021

(51) Int. Cl. A61N 2/00; A61B 5/00

(52) U.S. Cl. CPC A61N 2/00; A61B 5/4866

(57) ABSTRACT

The invention relates to the life energy spectrum derived from the natural world. Specifically, the invention disclosed a method for measuring a life energy spectrum (LES) derived from the natural world that can influence on humans and communication between humans and all things in nature, a method for utilizing a life energy spectrum (LES) that can influence on humans and communication between humans and all things in nature, devices and methods for generating life energy spectrum (LES) and curing diseases.

### CROSS-REFERENCE TO RELATED APPLICATION

This application claims the benefit of the U.S. Provisional Application No. 63/205,455 filed on Dec. 22, 2020 in the U.S. Pat. and Trademark Office, the whole disclosure of which is incorporated herein by reference.

### BACKGROUND OF THE INVENTION

### Field of the Invention

Embodiments of the present disclosure relate to a method for measuring a life energy spectrum (LES) derived from the natural world that can influence on humans and communication between humans and all things in nature, a method for utilizing a life energy spectrum (LES) that can influence on humans and communication between humans and all things in nature, and a device for generating life energy spectrum (LES).

### Description of the Related Art

The applicant has found an important part of the origin of life-Life Energy Spectrum (LES) from nature. It can be said that he has found the LES from nature. Due to the energy enhancement technology added by the applicant, intelligent functions have been added. Therefore, the LES of this application is better than the natural LES energy field. The scope of LES ranges from testing to analysis. The applicant believes that it is in the wide spectrum range of 0.2 micrometers to 15 millimeters. The accurate measurement of LES provides a very feasible method for humans to pursue and restore nature. There may be more significant advances in the field and communication between man and nature.

### SUMMARY OF THE INVENTION

The present disclosure provides a method for measuring a life energy spectrum (LES) derived from the natural world that can influence on humans and communication between humans and all things in nature, wherein the life energy spectrum is in a wide spectrum range of 0.2 micrometers to 15 millimeter.

A further embodiment of the present disclosure provides a method for utilizing a life energy spectrum (LES) that can influence on humans and communication between humans and all things in nature, wherein the life energy spectrum can realize effective regulation from the inside of living substances.

A further another embodiment of the present disclosure provides a device for generating life energy spectrum (LES), wherein the device can generate a life energy spectrum simulating a life energy spectrum derived from the natural world.

### BRIEF DESCRIPTION OF THE DRAWINGS

The above and other features of the present disclosure will become more apparent by describing in detail exemplary embodiments thereof with reference to the accompanying drawings, in which:

FIG. 1 shows the actual measurement diagram of LES.

FIGS. 2-1 shows that LES may use synthetic proteomics to identify the main proteins affected or related to the disease process.

FIG. 2-2 shows that LES can intelligently discover and find expression profiles, helping us understand where and how genes are expressed at the gene level.

FIGS. 2-3 shows that LES technology will intelligently search for, guide and regulate diseases genetically.

FIG. 3 shows a schematic diagram of the resonance effect of LES on everything.

FIG. 4 shows a schematic diagram of intelligent control negative feedback.

FIG. 5 shows that the LES + Ag&Cu Zelile technology is used in conjunction with its own zipper wound tape to accelerate wound healing and prevent infection.

FIGS. 6-1 shows a schematic diagram of LES + Ag&Cu Zelile + and rare earth element germanium-based negative oxygen ion integrated technology seed cultivation board.

FIGS. 6-2 is a photo of grass without LES treament.

FIG. 7 shows the comparison of LED treatment of AIDS and homosexual anal ulcers.

FIGS. 8-1 and 8-2 shows a picture of the basic healing of a 4th degree bed sore.

FIGS. 8-3 shows that the striated muscle syndrome of the legs has been treated with LES, and the ulcer has begun to dry out, and it is moving in a positive direction.

FIGS. 8-4 shows, after LES treatment, the striated muscle syndrome of the leg is basically gradually healed gradually.

FIGS. 8-5 shows that severe bedsores are close to healinge shows an old professor over 90, who miraculously survived and can walk alone.

FIG. 9 shows that LES can significantly improve red blood cell aggregation within 10 minutes.

FIG. 10 shows a local inferior cattle and its purebred New Zealand cattle baby.

### DETAILED DESCRIPTION OF PREFERRED EMBODIMENTS OF THE INVENTION

The applicant has found an important part of the origin of life-Life Energy Spectrum (LES) from nature. It can be said that he has found the LES from nature. Due to the energy enhancement technology added by the applicant, intelligent functions have been added. Therefore, the LES of this application is better than the natural LES energy field. The scope of LES ranges from testing to analysis. The applicant believes that it is in the wide spectrum range of 0.2 micrometers to 15 millimeters. The accurate measurement of LES provides a very feasible method for humans to pursue and restore nature. There may be more significant advances in the field and communication between man and nature.

LES enables all things on the earth to grow together and coexist without harming each other. Humans and nature rely on LES to coexist harmoniously. This is the ultimate goal pursued by mankind.

Due to accurate testing and finding a wide-spectrum LES from nature, it can realize effective regulation from the inside of living substances; coupled with human technology, the LES derived from nature adds more intelligent functions and can realize the state of life. The negative feedback control of all things in nature gives mankind the ability to find, regulate and repair the negative feedback balance from within the living matter and from the non-living bodies of all things on the earth. The applicant’s organism represented by humans has been tested and found to prove that organisms have a wide spectrum of energy radiation characteristics from micrometers to millimeters, and in such a wide spectforming a dream for intelligence in the field of materials that are mainly human beings and have broad spectrum radiation characteristics that react at the molecular level.

### Regulation

Applicants have discovered and are beginning to be widely used (LES) to make this dream possible. For example, every cell contains complete life information, and people try to communicate with nature from all angles from chemistry to physics, from qi to energy. The applicant believes that only by finding the scope of its life energy spectrum from the natural life energy of nature, which is the research object, is the only correct way for humans to study and communicate with humans and nature. For the first time in the world, the applicant successfully tested and found that the human body can produce a broad spectrum (Biospectrum Technology, BST) life energy spectrum (LES) from micro-millimeter waves. At the same time, it was discovered that the analog (LES) generator developed by the applicant earth needs LES to coordinate the entire ecosystem to support it to maintain balance. This LES ecosystem requires that all living and non-living LESs are as consistent as possible, and resonate together, are closely related to each other, and have a ring-to-ring structure. It is indispensable, just like human organs, the reason why people can live is to rely on LES to coordinate and supply energy to make each organ function normally. But the virus in the body is a threat to every organ of the human body. When it feels that an organ is uncomfortable, the human body has to take medicine or operate to kill the virus to ensure the normal operation of the organ. However, because people have not found effective control of the virus infection Method, so various viral diseases including AIDS are still difficult to deal with. And LES may control the infection hazard of the virus from the molecular level, from the aspects of cells, genes, and proteins, so that the virus can survive without harm. This is completely in line with the existing medical methods aimed at eliminating the virus. For the same reason, the earth needs to maintain the balance of the ecosystem. When the increase in human beings makes the earth feel uncomfortable, the earth or the universe will implement compulsory operations, including earthquakes, tsunamis, floods, droughts, hurricanes and other ruthless methods, which may be to protect the earth’s ecology.

### System Balance

Whether it is between species, or between humans and nature, or between humans, it is a community with a shared future. Mountains are high, and trees grow. LES enables all things on the earth to coexist and coexist without harming each other. Humans and nature rely on LES to coexist in harmony. This is the ultimate goal pursued by mankind.

There have been five mass extinctions in the history of the earth. The reasons are unclear. It has been recognized that we must do our best to protect the earth, restore the earth’s environmental ecology, reduce resource exploitation and be better for the earth. The applicant discovered and revealed that nature has LES, which may be an effective way to help people understand people and their communication with the earth. Only in this way can humans coexist peacefully with the “living body” of the earth.

LES brings the possibility of communication between people and the natural world. At this time, the applicant recalled that before the LES was not confirmed by the test, the famous physicists Wu Jianxiong and Yuan Jialiu had interviewed and instructed the applicant, and offered to take the initiative: willing to look for research to accurately measure the LES you proposed. This support is for the applicant That said, it couldn’t be more important. Moreover, the two great scientists also pointed out that it will definitely be measured. Once measured, it will be possible to promote the development of science and technology. The scientific community will refer to the measurement results and the theoretical explanations proposed by the applicant to introduce secondary innovations and make this discovery even more powerful.

In addition, the curves we researched and measured are continuous, which breaks the insurmountable gap between micro wave and millimeter wave in the past. The two great scientists, Wu Jianxiong and Yuan Jialiu, pointed out that the discovery and measurement of LES will mark that mankind has found the only standard for controlling health and survival from the natural world, which will have a huge impact on human society.

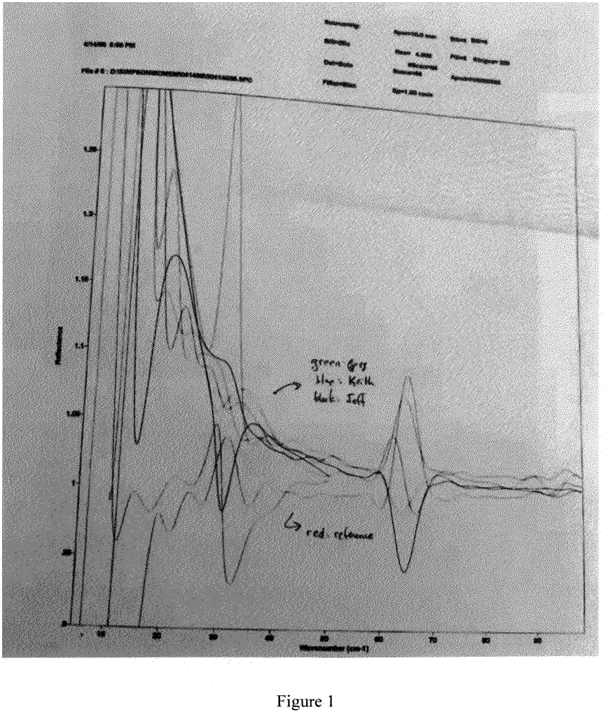

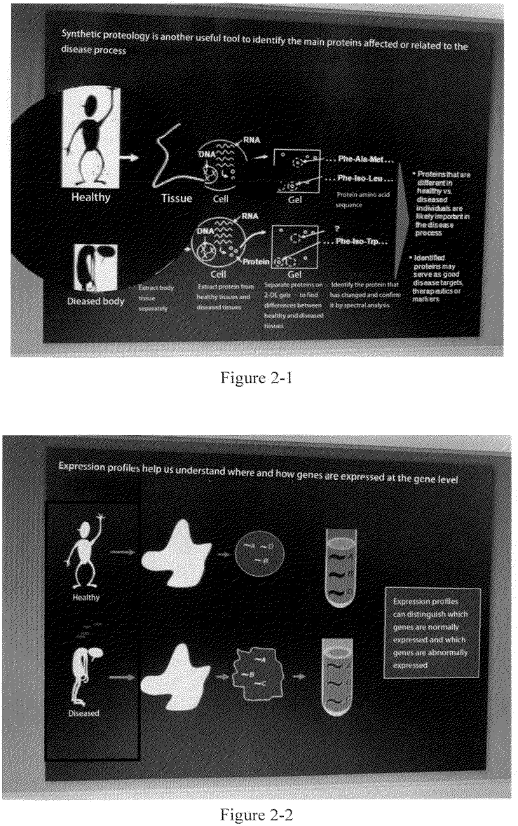

The actual measurement diagram of LES is shown in FIG. 1.

In FIG. 1, for the first time accurately (the life energy spectrum of each point, regardless of its frequency spectrum and amplitude) can be found from the test curve, which proves that the applicant has discovered and proposed the natural micro-millimeter wave Broad spectrum (LES) has established a good channel for human innovation and discovery to better help human survival and communication between humans and nature. The red curve in the picture is the simulation (LES) curve, and the other three curves are the (LES) curves signed by the three post-doctoral students in charge of testing.The similarity between the artificially simulated curve and the natural curve proposed and researched by the applicant proves that it has found a correct way to communicate with nature, which is amazing. Among them, the life energy spectrum curve of one graduate student is the opposite. The applicant found that the graduate student’s physique and spirit are weak. The applicant inferred that this may also be the one that (LES) may be able to distinguish between health and fitness.

The applicant has accurately measured the Life Energy Spectrum (LES) originating from the natural world, which provides goals and standards for the research and implementation of this patent. The key is how to make it very similar to this standard and become a technological issue. Using a variety of electronic oscillators, it is difficult to achieve such a wide-spectrum energy spectrum signal as LES. The applicant has been thinking for a long time, but starting from the vibration of all things, from why life energy comes from life, why all things in the world to rely on LES to maintain, what is the internal structure of humans and creatures, and understand all the structures. From this division, the applicant has found a method that is very similar to the LES in nature, and can take extreme. Complex problems are simplified, convenient and easy to use, large-scale production is large-scale, and easy to use in many fields. The amplitude and width of the LES can be controlled. This new type of wide-spectrum, high-power solid-state oscillator should also be quite versatile. The applicant introduces this production method in this application, so that interested parties can refer to this idea and model production. The production method used for LES is implemented according to the standard shown in FIG. 1. The applicant should point out that whether it is a partial or full realization of LES from nature, it will fall within the scope of this provisional patent and will be protected.

The inner influence of LES on everything

1. Carriers of nucleic acid genetic information

2. Protein expression of life information:
Its synthesis constitutes the main pathway of genetic information in organisms;
Its biological activity shows the necessity of life energy.

3. The characterization of biological information metabolism is the core of molecular biology research.

4. LES does not interfere with each other at the same time to the tens of thousands of reactions in living cells of the organism, cooperates with each other, and carries out their metabolism in an orderly manner, and is intelligently suitable for the organism in terms of synthesis, decomposition speed and quantity.

### Various Needs

The composition of the elements in the organism: Element composition: C, H, O, N, P, S and trace elements. Basic ingredients:
1. Protein, nucleic acid, polysaccharide, lipid
   One is also the four basic components of the organism, which act as the skeleton of the organism and provide energy. This is also the key to the role of LES in the molecule.
2. Vitamins, hormones, organic acids once biomass. Among them, enzymes, vitamins, and hormones are catalytic regulators. LES also has a great influence on these three.
3. Inorganic salt and water
   Common inorganic salts K, Na, Ca, Mg salts, etc. Because LES is derived from nature, it has obvious replacement and repair effects on the three basic components in these organisms.
   LES also plays a very important role in the intelligent repair of cells, genes, and proteins in organisms.

Since LES studies the existence of life at the molecular level, we have also discovered the wide-spectrum characteristics of biology by our research and testing. With the help of humans, it is natural to discover the junction of biology and physics, which is a very important one. It was discovered that the future will bring breakthroughs in the field of life, especially in the field of medicine, and make some of the possibilities of the existing medicine that viral infectious diseases are difficult to treat as the past.

Since LES searches for and repairs upward or downward abnormalities from the above three and genes, proteins to intelligence, it has an important role in keeping organisms in balance, so it should have obvious replacement and repair effects on various diseases. Therefore, LES medicine and life sciences have begun to emerge.

Further research and application of LES reveal

Using the intersection and combination of LES and various existing disciplines, we have reached a new field and perspective for understanding life, making it more diverse.

It further reveals the nature of multi-disciplinary summary findings such as the physics and biochemistry of life.

Develop new methods of life research and transformation.

In meditation, I feel that there is a mysterious power that inspires people to anticipate the entire grand universe. It turns out that this mysterious power is our brain. The human brain, which is actively and constantly creative, is estimated to be one of the most valuable forms of energy in the universe. The magnificent universe, the human beings who are restricted to survive on the earth are so short-sighted. The brain that constantly exerts creativity is the hope for humans to recognize the universe. The universe is not simply nothingness, nor is it an unrecognizable grandeur. The mysterious powers are not the human form of Allah, Jesus, and Buddha, but may be electromagnetic waves, LES energy, or multidimensional energy forms that the human brain cannot recognize temporarily. Living a hundred years, looking at 10,000 years, the human brain’s civilization inheritance is too slow. There must be simple predictions. Once the human brain dies, it stops cognition. This world does not exist for it, so it does not exist anymore. If we abandon the main shaft, a new type of human brain that relies on the eternal existence of other forms and has a steady stream of creativity appears. This shows that human beings are only a very small form of energy existence in the universe, and LES must have a coordination and balance role in it.

Each user is a small universe that contains all life information, including wars that lasted for billions of years, spaceships. The applicant transferred to the cell world described in the BBC classic record “Cell’s Inner Journey” to increase the intelligent auxiliary role of describing the energy spectrum of life in it. Every moment, there is a big battle going on in your body. This story originated billions of years ago about human cells and adenovirus fighting for control of our DNA. Fighting is still happening in each of us today. This is the story of virus invasion-the battle of cell life and death. The film of LES transfer reveals the fine mechanism of the human cell system from the inner world of the cell: from the fanatical, the cell membrane that acts as a security defense system against every object that enters and exits the cell, the cell frame that transfers the cell’s substance particles, and the mitochondria that keep the entire cell world in motion, to the cell nucleus that preserves DNA and thousands of smart LESs each originating from nature, can indeed inhibit viruses and by transforming the DNA, proteins, and the security defense system of each object in the cell. The infection and destruction of germs, without the heavy blow of life and death, gently let the virus and germs coexist peacefully with the body under the replacement of loss of destructive power. It also avoids that the existing therapies often use cruel treatments that cannot completely effectively eliminate the virus, causing the virus to dying and struggling to produce a huge virus, which ultimately brings more serious consequences to life.

The applicant believes: this latest discovery of cells, this thrilling record film brings us into the gorgeous inner world of human cells for the first time, and reveals to us a cutting-edge and intuitive biology that has never been involved. It also indirectly reproduces that LES intelligently assists in participating in each object in the cell, each running serial activities, and helping human beings in the multi-billion-year war during this period, making each object in the body more. It not only inherits the true origin of life well, but also can quickly transform well to resist and adapt to the ever-changing living environment.

Cells are the basic structural and functional units of living organisms, essentially a small universe.

From the perspective of biological structure, (LES) is applicable to all living beings and everything in the world, including medicine, agricultural plant industry, animal husbandry, and the space used by humans has become a major transformation similar to the natural environment and the entire life sciences, because It is the life energy spectrum derived from the natural world, so it has accurate and wide-ranging uses for all things in the natural world of the world, and it will bring unprecedented revolutionary changes from within each creature or one of all things.

The sensitivity of living beings to the external physical information field should be similar to that of the physical energy field and the biological energy field of the biological energy itself. The more similar, the easier it is to produce sensitive reactions. The more beneficial it is to the regulation and improvement of biological life processes. For non-living substances, as long as they can respond to the effects of the broad spectrum of life energy, they can also be adjusted to affect these substances to produce obvious changes. (For example, the entire chemical reaction is completed by treating methanol with the life energy spectrum for 30 minutes. With the special equipment for handling methanol chemical reaction, the methanol reaction just started after 3 hours. It shows that the life energy spectrum can be used for chemical engineering in chemical engineering. The reaction has a revolutionary effect). The earth we live on is just a suspended particle in the universe. Although it is a very small dust that is difficult to find, it also shows the importance of life.

The treatment of BST is completely different from traditional medical treatments. BST is a natural therapy that seeks and repairs diseases intelligently from the aspects of cells, genes, and proteins. However, traditional medical methods are mostly based on removing the surface cause of the disease. Different methods have completely different results.

The applicant believes that living and normal life information should be the subject of research. Existing medical research mostly uses in vitro or even inanimate tissues, cells, genes, proteins, etc. as research objects, which are difficult to obtain accurate results.

Since the development of science, the great significance of quantum mechanics (which is considered to be more accurate as quantum science) has been proposed and discovered, and quantum science is better at studying the microscopic state. In fact, when human beings were born, there were thoughts (thoughts), and the soul of humans was born at the same time, but it was invisible and intangible with the naked eye, which attracted enough human research and attention. Just imagine, if people don’t have energy, they are not just vegetative people like zombies. So quantum science is also a science that studies the human soul, the science of human spirit. Essence is derived from the soul, from the energy and information support of LES. Where does the wisdom of man come from, where does the extraordinary performance of man come from, where does his amazing perseverance come from, where does his dream come true. It all comes from the soul of man and the role of man’s LES.

LES technology can also be used in social management. It is a science that helps to research and solve extremely complicated social problems. It allows the government and the public to resonate with LES, which is conducive to the government’s efforts to improve the needs of the public and let everyone LES. Socialization may be continuously improved by the government and researchers as a new theory of social management. The public appreciates that wise men have formulated some necessary regulations in advance to keep private property, especially technological innovations under strict protection, which promotes social progress and development. It is believed that in social development, leaders and the general public are both important. From the perspective of LES research, the choice of social leaders is no longer compatible with the rapid development of today’s society. I believe that we can consider combining LES research with big data and purposeful test conversions, and intelligently screen many candidates, and discover the real young and powerful, full of wise leaders. The leaders must leave after their term of office. At the same time, we also choose to enrich think tanks and people with different opinions, and adopt reasonable and effective methods such as LES social research and big data to analyze and monitor the leaders at any time. If the behavior of the leaders is found to have crossed the red line, the candidates can be replaced on the stage.

To form an effective assistance and supervision mechanism.

It saves a huge expenditure of money, the antagonism among the public. Applicants do not understand social management, but only put forward their own ideas from unexpected research ideas.

LES is used by the United Nations to coordinate the pursuit of peace, development, and prosperity for all countries. LES will resonate with each other for achieving this grand goal. There will be no market for political, territorial, and interest disputes between countries. A true global village in the world will appear under such a reasonable system. Peace, freedom, and health are the only combination, and the world will become a true global village.

LES also has an intelligent endogenous heat effect, in addition to decomposing the circulatory system in the body, keeping it open. It can also participate in the repair of cells, genes, and proteins when there are deviations to help them restore their health.

Because LES has a wide spectrum from micrometers to millimeters, while microwaves produce thermal effects, millimeter waves produce non-thermal effects. In this complex wide-spectrum environment, non-thermal effects appear in it, which makes the entire life system. LES plays a better role in intelligent balancing.

Both endogenous heating effects and non-thermal effects are one of the characteristics of LES.

In addition to the usual research from tissues, cells, genes, and proteomics, (please note that most of these basic researches depart from life forms and life energy spectrum fields, and it is difficult to reflect their true conditions). It should also include aspects of the living whole, such as aura, field, and state of life, because only the information of living life energy can reflect the most true state.

Human intelligence is improving, but human body constitution is deteriorating. Human basic body temperature has changed from about> 37 degrees in the 60s and 70s to about> 36 degrees now. The body temperature has dropped by nearly one degree, but it has brought countless things to humans. The endogenous and non-thermal effects of LES can help people raise their body temperature, resist the erosion of disease bacteria, and maintain people’s health. It can be said that LES energy = health, health = LES energy. The understanding and balance between man, man and nature is very important, because it maintains the existence of life systems and nature.

Life forms and everything in the world have different levels of giant systems or small systems. Giant systems like living bodies are very complex and changeable. The existing medical system can only conduct research on a certain disease one by one, and develop specialized drugs for this disease. It is only a general treatment for the population of this disease and cannot be intelligently targeted for each patient. Diagnosis and treatment, special treatment for each person’s changes is a dream, and it is impossible to realize it. However, the (LES) that the applicant has discovered and applied can intelligently find abnormalities in cells, genes, and proteins, and also intelligently repair abnormal replacements. It can be intelligently targeted for each person’s disease. This should be the main purpose of the development of modern intelligent medicine.

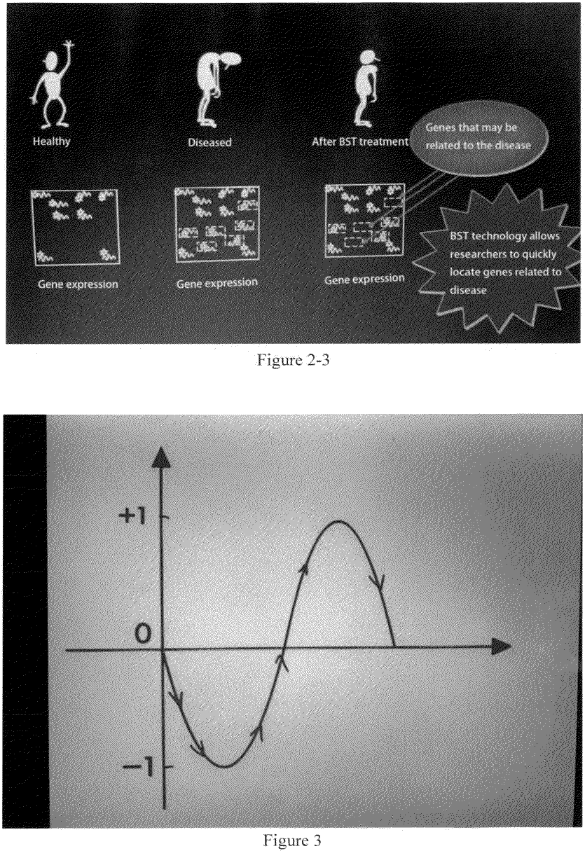

The applicant found that because LES is derived from LES in nature, and the supplementer has increased the energy of smart LES, it can conduct intelligent inspections on cells, genes, and proteins. After discovering abnormal deviations, it will mobilize the immune system. The research and development of the application of this intelligent alternative system to the life system will enable these research objects to appear well expressed and coordinated, making complex life science research easier and more colorful. Including the intelligent transformation of life from the genetic and proteomic level research can also be clearly discovered and discovered. All this is of great significance. The applicant cooperated with the AIDS laboratory and found less than 10 digits of AIDS-related gene changes using LES. In cooperation with the Leukemia Research Center, we found less than 10 changes in leukemia-related genes. In cooperation with the university’s laboratory in animal research on the treatment of optic nerve injury, it was discovered that heat shock protein 27, mainly heat shock protein, was involved in the repair. By analogy, the applicant uses LES to treat various diseases, and similar disease-related genes and proteins participate in transformation and repair. It is believed that LES can be used to conduct animal experiments on various diseases, discovering genes and proteins related to various diseases one by one, and compiling these results into a manual of diseases and genes and proteins for doctors, pharmaceutical companies, and laboratories. Refer to when researching, it may cause. See attached FIG. 2.

FIGS. 2-1 shows that LES may use synthetic proteomics to identify the main proteins affected or related to the disease process. Another nine tools. FIG. 2-2 shows that LES can intelligently discover and find expression profiles, helping us understand where and how genes are expressed at the gene level. FIGS. 2-3 shows that LES technology will intelligently search for, guide and regulate diseases genetically.

The applicant uses scientific and technological methods to increase the energy level of the life energy spectrum derived from nature, and maintains the original ecological life energy spectrum information of the nature, which will enable the life energy spectrum derived from the natural world to have the intelligent ability added by humans. It is formed not only from the natural world; but also with human intelligence, which will have a very significant effect on humans, the natural world, and the communication between humans and the natural world. This is the ultimate purpose of the applicant’s application for the present invention.

LES has the following three characteristics: 1. The life energy spectrum derived from nature indicates that humans research and apply natural essences from the natural world, and use the essence of nature for all aspects of human survival and life. 2. The study of living organisms makes (LES) the study of life closer to the true state of life. 3. Add artificial intelligence technology to make (LES) more intelligent and accurate in finding and repairing problems for life and everything. Taking humans as an example, any discomfort or disease in the body is a positive or negative shift in the basic particles of cells, genes, and proteins in the body. The LES will intelligently find and regulate these shifts through the immune system. Repair, help them to return to normal 0 state. In this way, LES has established an intelligent balance regulation system in the life system to solve problems from the root causes of diseases. This is also the fundamental reason why LES can regulate and repair various diseases. Humans dream of sickness without drugs; no side effects; the real natural therapy that intelligently finds and regulates diseases begins to become a reality in humans under the action of LES. The applicant believes that humans can use intelligent (LES) to communicate with the natural world to influence and assist the natural world to develop better. These three articles constitute the main core content that (LES) has an important impact on mankind and all things in the world.

Based on the findings (LES), we conceive and conduct research to understand that the life and life energy produced in nature will bring wonderful effects. Will intelligently influence and regulate any life very effectively. The applicant believes that this is also a movie that allows people to appreciate the wonder of you and the creatures and is becoming more real. Life is responding to you, communicating with you. There are no accidents or coincidences in life: every object has its own unique (LES). When anything enters your life, it means it is on the same (LES) as you. Everything you see-the contents of each sign, the font and description of the text, sounds, colors, people, objects, etc.-everything you hear, and every situation and event, such as with you (LES) When you agree, you will have an attitude of approval, pleasure, and appreciation from your heart to your expression. The understanding of (LES) should be broad, multi-angle and all-round. The applicant believes that the broad spectrum of the LES discovered and invented by the applicant is derived from the LES in nature. The broad spectrum characteristics of the LES discovered and invented by the applicant are far superior to people’s speculative understanding of the role of nature. The applicant organized and implemented the LES in this application. The biological impacts of the LES in this application range from large to spatial biological fields and small to genes, proteomes and possibly even infinitely small particles. This has produced countless pasts on the human body and organisms. Functions and effects that people can’t imagine. Therefore, the applicant believes that the wide frequency spectrum of LES has unlimited impact on organisms, and its regulating and repairing effects on human bodies and organisms are also endless. Applicants have overcome many critical illnesses by themselves and realized that human beliefs are very important. In fact, human beliefs were born at the same time as human beings. It is just that beliefs are manifested and evolved from a microscopic perspective. People are invisible, intangible and often ignored. Mankind discovered that to this day, Einstein’s comprehensive development of matter/energy/time/space/information has only completely described the material world. Now people begin to understand the whole life system from the microscopic, from the aspect of quantum mechanics, and gradually study “consciousness” and the realm involving soul. If you are confident and will not give up even when you are critically ill, this positive energy belief will take care of you, produce beneficial hormones that help you overcome the disease and all other changes that help you overcome the disease, and ultimately make you immune. The system and other systems of the body are getting closer to normal. This is to have positive energy beliefs, pray for God’s blessings, and persist in helping others to do good deeds for a long time. It is possible that you will be born again and live again. Of course, some patients with advanced tumors said that they must build their confidence, but when they saw the terrible test reports again and again, and heard the doctor admit that they were not good, his belief was defeated, and his life would quickly end and become his only option.

The qi mentioned here is a kind of energy that sustains life. This energy produces all things. Wherever there is life, or even where there is no life, there is this kind of energy. Everything in the world is composed of these energies. This shows how important the natural LES discovered and applied by the applicant is.

What is a person? When qi gathers, it is born. There is a predestined relationship, and we are together. The five types of Qi-gold, wood, water, fire, and earth come together to form a LES field, and there is life. If the gas is dispersed, it will die. As the saying goes, suffocation, the qi that does not breathe is the energy of the world. There is no such qi to support you, which leads to the end of your life. Therefore, humans must realize the importance of this Qi, the LES that originated in nature.

Qi, including the energy of the LES field, should also be the Qi of the universe. It is definitely not reflected in the breath, but in all things and everything. Moreover, the energy of this qi is very large. Imagine how heavy the heaviest thing around us is, and this qi can move. The mountain is heavy, qi can definitely push him, and there is nothing more that can push him. Everyone sees that earthquakes can move mountains and fill seas, and the energy is big enough. Because no matter how heavy the mountain sinks, the earth will not sink. What is the state of the earth? The ancients said: the grandeur. From the perspective of the universe, the earth is as small as a ping-pong ball floating in the water. Since the air of the universe can lift up the earth, what a big mountain is, as a human being, it is nothing more. And wherever this qi goes, it controls everything. Finally, you will also find that agriculture, animal husbandry, flowers and trees are also inseparable from energy. Without the energy of the LES field, there would be no agriculture; without animal husbandry, without medicine, there would be no life, without human beings and all things in the world. So people have to ask what the LES field can be used in. The applicant believes that it should be omnipotent. Because it is the LES from nature, this is the root of its application to all things.

Dao is simple, doctor is simple, one yin and one yang is the way, yin and yang balance all diseases. These two sentences are very important. They are the core idea of maintaining life and prolonging life. LES will intelligently assist the life balance system to correct the deviations in cells, genes, proteins, and basic particles that cause disease discomfort, and help the body’s five internal organs to achieve a state of yin and yang balance, so many diseases in the body will disappear by themselves. The body has the function of adjusting the balance for itself like animals and plants, but because of the advancement of human medical technology, people’s self-healing function is passively dormant, and it needs to artificially try to activate and remind the body’s immune system. Play an important self-healing role in order to survive. The applicant’s research proves that the life energy spectrum can provide people with no drugs for sickness; no side effects; intelligent search and regulation of natural treatments for diseases, which is an important system for intelligently controlling diseases and maintaining life balance. This may be an important supplement to medical development.

If yang refers to the sky, yin refers to the earth. If yang refers to people, yin refers to ghosts.

If yang refers to men, yin refers to women. If yang refers to day, yin refers to night.

If yang means rigidity, yin means softness. If yang means positive, yin means negative.

The concept of yin and yang is derived from the natural view of the ancient Chinese, and the observed natural phenomena, such as heaven and earth, sun and moon, day and night, cold and heat, men and women, upper and lower, etc., are summarized in a philosophical way of thinking. It also shows that everything in nature depends on balance to survive.

Therefore, the importance of the energy of the LES field is reflected in all things in the world. The parents have given life to the LES field. In the end, the person who wants to survive depends on the LES energy to continuously supply him. This person grows by absorbing energy continuously. Whether it is grass or animals, they grow up with the energy supplied by the LES, and everything in the world grows with the energy delivered by the LES field.

(LES) No matter you are in any state, you can eliminate all kinds of interferences, help you calm down, and still be full of confidence to survive when faced with dangerous indicators and life crises. The miracle of life is often. When your life is dying, the warm sunshine appears brightly and brilliantly in your heart, shining on your body, and your chance to live again is presented to you again. Why are there so many believers in various religions (including Buddhism, Christianity, etc.) to worship the faith? This also includes some content related to the life information field. That is, the effects of essence, energy, and spirit are also very important to health. Gravitational waves have been discovered many times since 2015. The discovery and confirmation of gravitational waves will have a huge impact on the physics of the world. Many thoughts that were impossible became possible with the discovery of gravitational waves. With the help of the discovery of gravitational waves, there will also be a new improvement and understanding of the research and application of the LES wide spectrum. The applicant for the patent of the present invention believes that scientists can help research and use it to summarize some new discoveries, inventions and applications. We found that inventions and experiments have proved the natural LES field from the micrometer-millimeter wide spectrum generated by the spontaneous radiation of living organisms. After we apply intelligent control technology, mankind obtains a life energy spectrum that originates from nature and is superior to nature. Using this life energy spectrum will affect the entire natural world and the realm of life. Including medicine, agriculture, animal husbandry, life sciences and all biological and non-biological things in the world whose internal changes are affected by (LES) related fields. It will bring revolutionary changes to the field involved, which is the purpose of the invention patent of this application. You can use attached picture 3 for demonstration. FIG. 3 shows a schematic diagram of the resonance effect of LES on everything.

The frequency spectrum in physics is the number of vibrations per unit time. In physics, the number rate of vibration is called Hz (hertz) or cps (number of cycles). Normally Hz is calculated in seconds. One vibration per second is 1 Hz, and one thousand vibrations per second is 1,000 Hz. 1,000 Hz =1 KHz (kilohertz) 1,000 ,000 Hz = 1 MHz (megahertz) 1,000,000,000 =1 GHz. The above-mentioned researches are limited to static physics research and do not involve endlessly changing life. The applicant of this patent discovered that humans and organisms have a biological energy spectrum derived from nature, between the broad spectrum of micro-millimeter waves, and has gone from the discovery of the original megatrend to the laboratory’s first accurate test discovery and confirmation in the world Both the human body and the LES instrument that simulates the human body can generate micro-millimeter wave wide spectrum characteristics. Let’s not talk about the important effect of LES on earth creatures and all things. This test alone proves that the junction of biology and physics has been found by humans. Among them, the use of medicine for human health care is superior to the existing treatment methods for sickness without drugs; no side effects; and the revolutionary function of benign repair and regulation on diseases is called life spectrum medicine; used in agriculture, in terms of yield and nutrition Ingredients, taste and to maintain and repair the original genes, so that humans can truly eat pure natural food, improve and improve the level of human health and survival of the revolutionary life spectrum agricultural science; fundamentally improve the applicant’s discovery and invention Poor breeds of animal husbandry and proliferation of diseases can help the world to become better varieties and disease-free. It has unique breeding technology for those endangered rare animals and birds, and it also has problems with infertility faced by humans. Plays a major role. The applicant for the patent of this invention calls it the life spectrum animal husbandry technology.

The applicant of the present invention has discovered: universe, galaxy, solar system, planets, earth, land, sky, ocean, human beings, animals, plants, music, musical instruments, radios, clocks, brain waves, AC and DC electricity, biological mating, hypnosis, mind power, Text, color, laser, language, heartbeat (Hz)...all contain specific life energy spectrum directly and indirectly related to it.

FM radio 103.5 MHz = 103,500 ,000 Hz, that is, FM radio transmits the broadcast content to the air with an electromagnetic wave frequency that vibrates 103,500 ,000 times per second, and then is received by the radio of the same frequency. The transmission and reception must be of the same frequency. The applicant of the present invention believes that if the broad-spectrum waves of the LES originating in nature are used as communication signals, the connection between all things in nature and the connection between the earth and the universe should be completed by the LES. Therefore, understanding, applying and using LES for communication will also bring breakthrough progress in the field of communication. The same LES will produce the “resonant LES effect.” Because of its unique and varied biological resonance and harmonious LES effect, people may obtain new means of communication.

Life energy = life

Without life energy, there is no life, which shows how important the LES is to life.

Resonance is the only way for everything to communicate with each other through resonance. Therefore, the applicant believes that it will be very meaningful to use this method of communication between all things relying on the resonance effect to establish a new communication method for mankind. The universe was originally created by vibration (Big Bang Theory). The void of the universe, and even the Milky Way that exists as a physical image in the space, are all born from the vibration of LES. If God exists, then the so-called God should be LES. LES is the soul of tangible matter. Without LES, tangible matter will become void. Everything on the earth is no exception. The natural LESs of stones and tree roots are different; the LESs of gold, wood, water, fire, and earth are also different; the LESs of human organs, nerves, and glands are also different. All vibrations can only be achieved in a harmonious state. Stability and balance. It is also different; the LES of human organs, nerves, and glands are also different. All vibrations can be stabilized and balanced only in a harmonious state.

### Life Energy Spectrum and Health

When it comes to the structure and system of the human body, you can think of it as a small universe in the big universe. From the beginning of conception (the LES is wrong, you will not get pregnant), the development and growth of human beings are gradually developed in accordance with the principle of intergrowth of organs. If the LES of the upper organs is wrong, the lower organs will not be produced, or the growth and function of the lower organs will decline. The human body’s heart, liver, lungs, kidneys, nerves, bones, cells, and DNA each hold a specific LES, but they can work in harmony with each other in resonance. If you imagine our body is like a large symphony orchestra, performing a beautiful movement in a harmonious way. When a gland, organ or cell starts to lose its LES, it will not be able to resonate with other group members around. If this phenomenon continues, the body will have serious health problems.

How to use the LES resonance principle to restore the normal operation of various organs of the body is the principle of LES application in physical health.

### The Scope of Human Application on the Inanimate Spectrum

In the 20th century, human beings have made rapid progress in the development of frequency applications, from the invention of radio to satellite navigation of space technology; from computers to quantum computers; from gunpowder to microwave weapons; from spectral analysis to dimensional conversion, future applications and developments Part of it is too broad and too broad. In the 23rd century, robots or third-type life forms created by LES may have taken the helm of the earth. From the perspective of human application in LES, it can be roughly classified as follows:
Military-infrasonic waves, star killers, satellite communications, navigation, thermal missiles...
On the ocean - Sonar (deep sea exploration), cultivation and development of marine life, fishing...
Education and training-mental and potential training, preschool education, concentration training, and even intelligent and fast input of various professional skills, language, thinking, etc. into the brain, quickly allowing people to become experts in various skills from a layman.

Medically-Ultrasound, X-ray, proton therapy, LES have natural medical therapies that keep the life system in balance.

Architectural — All building materials can be added with LES technology, so that the indoor environment has a fresh, healthy, harmful bacteria-free air and a vitality field similar to the natural environment.

Exploration and communication-ocean, mineral vein, communication, telegraph, telephone, remote control...
Industrial - cutting, ultrasonic...
Agriculture and animal husbandry-crop growth, variety improvement, pest control...
Religiously-spiritual practice, supernatural powers...
Meteorologically-weather forecast, change...
Commercially-communications, optical fiber technology...
Optically-LED technology, spectrum analysis, laser...
Others-Discussion and application of fractal theory, butterfly effect, holographic theory...
LES is the essence of the universe. It is filled with all entities and intangibles in the physical and intangible worlds. When people try to understand and apply its characteristics, don’t forget that human beings are also its products, so the original parents of humans can It is called LES because it is the source or creator of everything.

The effect of LES on disease and rehabilitation and longevity.

Enzyme molecule is also a type of protein molecule. No matter which kind of enzyme, its structure has the same molecular skeleton. When it is affected by the life energy spectrum field, it will enhance the vibration of amide-I and amino acid residues, thus making The activity of enzymes and the ability to participate in and promote chemical reactions are strengthened. In addition, because LES can restore the ability of protein molecules or cells that have been invaded or corroded by impurities, viruses and bacteria to a normal state of movement, the effects of impurities and viruses are eliminated. On the macro level, it is manifested as enhanced immunity.

The impact of the energy spectrum of life on the molecular level. The vibration frequency of these molecular chains and the relative vibration frequency between the molecular chains. The results show that, considering various types, they can absorb electromagnetic waves in far infrared or microwave bands such as λ = 0.14-0.2 mm or λ = 0.78 mm. Different protein molecules have different molecular weights (some are very different), and other protein molecules are much more affected than the molecular weight of α-helical proteins and the surrounding molecules and the environment. From infrared to microwave bands, it will also be possible to broaden the wide frequency spectrum. Therefore, the applicant’s actual measurement found that the absorption wavelength is longer and wider than the research and analysis wavelength.

We would be very surprised if we compare it with the spectrum generated by the life energy spectrum discovered by the applicant. The two are very consistent, that is, the broad spectrum of life energy discovered by the applicant also has peaks in the 0.72 µm-8 µm band, and there are also broadband in far-infrared and microwaves, and there is a band wave field, Which is completely consistent with the life energy spectrum derived from nature. This coincidence is not a coincidence. It tells us the fact that the electromagnetic waves radiated by the life energy spectrum field discovered by the applicant are resonantly absorbed by protein molecules. After the protein molecule is absorbed, it will cause or maintain the C═O bond. The vibration of amide-I and the vibration of the molecular chain or amino acid residues makes the vibration quantum of amide-I and the vibration of the molecular chain usually couple to form a soliton, thereby normally transferring biological energy along the protein molecular chain to make each tissue normal Exercise and muscle and cell activities have normal biological functions. If it is a patient or organism, it will absorb the biological energy spectrum for those diseases that are hindered by impurities, viruses or bacteria or external fields. After the transmission spectrum, it will be affected by the amide-I vibration and part of the molecular chain. It can be restored to normal, so that bioenergy can be restored. In order to transfer the normal state, the disease can be eliminated. In this sense, the wide frequency field of the LES spectrum will have a significant effect on disease and virus infection.

The main effect of LES energy spectrometer on living organisms is to start from the molecular level. It stimulates and activates the internal excitation of protein molecules and the vibration of the molecular chain, so that the biological energy can be transmitted normally, thus improving the vitality of life, and recovering the virus and impurities. It erodes the body and eliminates the disease. This shows that its main function is to regulate and mobilize or restore the movement of protein molecules and their atoms, molecules and electrons to a normal state, thereby eliminating viruses, impurities and other poisons, rather than directly killing the virus. This is obviously different from killing. Existing medical methods for viruses and bacteria. The applicant found that the life energy spectrum is a natural therapy that treats diseases without drugs; has no side effects; intelligently finds and controls diseases; it is a way to intelligently maintain and regulate the balance of the life system from the LES that constitutes the life body. It is a natural therapy composed of natural LES and intelligent adjustment functions added by humans, which can treat diseases and maintain health. It can help people all over the world use it comfortably every day. It will also bring huge adjuvant treatment and health effects to the United States.

This kind of LES+human discovery from nature and the cooperation of science and technology can be applied to the medical field of humans, and it is possible to achieve no drugs; no side effects; intelligent search and treatment of various diseases can be summarized in real natural therapy From the perspective of human health, this will undoubtedly bring about a revolutionary change, and the global public can easily understand and master the treatment and health care of this LES for themselves and others. As long as they insist on using it, the metropolis will gain even An effect that even a big doctor can’t get. Even for viral diseases such as the new coronavirus, which is currently raging around the world, due to its continuous mutation, it will be very difficult for the existing medical community to develop vaccines and treatments. For viral diseases such as AIDS, people have not really effective until now. method. Long-term coexistence with these viral diseases will become normal. However, the LES technology of this application may break this frontier. LES can control the activities of the virus at the molecular level and make it incapable of infection. A new situation in which humans and viruses can coexist peacefully without any harm can appear. And it saves the human beings struggling to find a virus vaccine. Various viral infectious diseases such as pneumonia, hepatitis, diarrhea, influenza, lupus erythematosus, herpes, and viral keratitis are beginning to show new approaches and new curative effects that may be superior to existing therapies.

The molecular level allows the virus to be controlled, and it may help solve the situation where the existing medical methods are helpless against the new coronavirus and other viral infectious diseases. It is expected to present advantages in terms of effect and cost.

LES is a wide spectrum from micrometers to millimeters, which is well consistent with LES in nature. This is not accidental, but a major discovery made by the applicant from science to practice. According to the regulation response and clinical effect analysis of the cell, gene, and proteomics of the intelligent wide-spectrum feature of LES, it is found that LES regulates the virus at the molecular level, so that it loses the ability to infect, but it can coexist with humans. This is completely different from the conventional medical methods and thinking of trying to kill the virus.

### Intelligent Regulation of Biological Spectrum

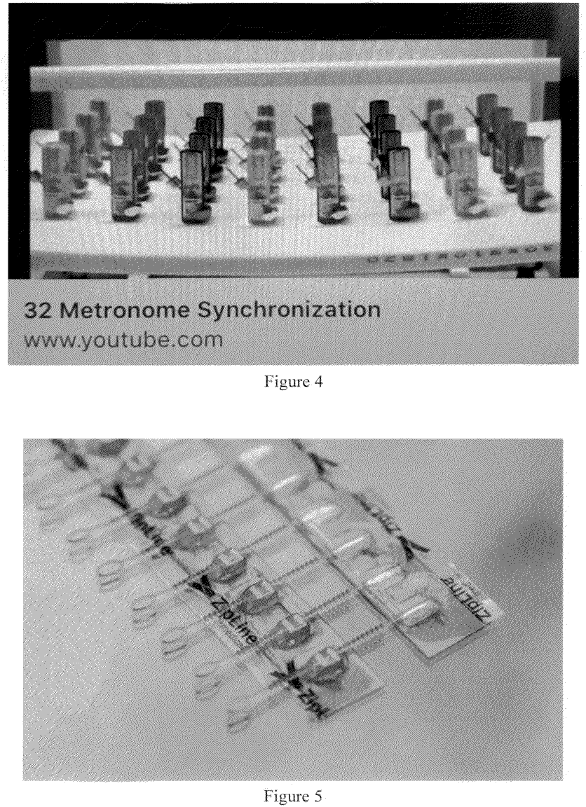

For the biological-dominated field, it has an unprecedented healthy coordinate with 0. It will intelligently look for positive deviations that develop upwards, and develop downwards into negative deviations. After finding them, they will intelligently adjust the positive and negative deviations to the trend. A health coordinate close to 0. (See FIG. 4). FIG. 4 shows a schematic diagram of intelligent control negative feedback.

Since the birth of mankind, in addition to the macroscopic body, the microscopic spirit has also been born, and they have been developing and progressing every day. LES has an important influence on the human spirit. People have given many definitions of “halo”. The actual halo is the energy field that exists in all matter. Whether it is people, animals, or even trees, stones, and other things in the world, they all have their own aura. Therefore, LES has an important influence on everything.

The generation of the halo must be traced back to the atoms that make up the matter. Atoms are composed of protons and electrons. This is electromagnetic field motion, and living bodies are more active than non-living bodies. Therefore, the aura of plants, animals and humans is easier to observe.

The human aura is an energy field surrounding the body. It centers on the human body and radiates in all directions. The energy field of a healthy person forms a standard ellipse or oval around the body.

Although we have not been able to find out how strong the aura of some great men is, there is no doubt: the healthier your body and spirit, the more active your energy will be, and the further the aura will spread. Moreover, the more your aura is fully stimulated, the more you need to do what you want and do. Similarly, the more promising your career is, the more you will suffer jealousy, slander, including theft and forgery. The stronger your aura, the more you can restrain the interference of unfriendly energy from the outside world.

Human body luminescence is also called “human body flame”, “spiritual energy” and “aura” (Aura), which is actually a human aura. The glow of the human body belongs to object light, similar to “cold light” and does not generate heat. It has a major impact on the existence of human life. A person can roughly judge a person’s state by the color and intensity of the human body’s luminescence, its morality, health, mood and luck.

Everyone has brilliance, but they are different. The light emitted by ordinary people is only about 20 mm, which is not easy for human eyes to see under normal circumstances and conditions. However, qigong practitioners, especially those who are well-trained in qigong, emit a strong light. When they reach a certain power level, the glow can appear as visible light in the dark, and most people can see it with their eyes. Observe the developed workers carefully during the day, and you will also find their light, such as the “burning” air heated by the sun on the asphalt road. Glow is composed of biophotons emitted by the human body. Biophotons are small particles of light emitted by living organisms. The interference between photons, diffraction, standing waves and absorption constitute a communication system between living things.

The main glow of the human body is usually located directly above the head, like the flame of a candle. The luminescence is divided into three layers, and some extend to two feet. The light of ordinary people is monochromatic, with up to three colors, and the diameter of the halo can reach 30 to 50 cm. This halo emits energy, just like the flame of a candle emits light.

Manual workers or people who like to exercise are more glamorous than mental workers; young people’s body surface luminescence intensity is twice that of the elderly, while the luminescence intensity of the elderly is the same as that of the young people.

LES fracture accelerated healing support series. In the past, plasters or braces were often used for fractures, allowing the callus at the fracture site to recover naturally and slowly, which took a long time and was painful and inconvenient for the patient. Adding LES technology to the support can improve fluid circulation, repair the nervous system, and accelerate the formation of callus. Generally, the callus at the fracture site can be completely formed within 2 to 4 weeks, and it is significantly shortened.

### The Healing Process

The distribution of human body luminescence also has certain rules. Under normal circumstances, a person’s fingertips glow the strongest, while the arms, legs, and torso are weaker. The upper limbs usually emit lighter than the lower limbs. Although different parts of the human body are closely adjacent, the intensity of their glow may be twice, twice or even ten times different. The luminous intensity of the same part is always maintained at a certain luminous level until some special changes occur in the life state.

LES water functional nutrient solution, a life energy-rich liquid processed by LES technology + Ve, VD, + ginseng, panax notoginseng, dendrobium, etc., combines the advantages of the East and the West, can improve the immune function, adjust the circulation and nervous system, and relieve fatigue Symptoms, enhance people’s vitality, help calm the mind and help sleep.

When the LES produces a balance regulation effect in a three-dimensional space, the applicant also thought that it could consider proton heavy ion therapy techniques like gamma knife and the most advanced radiotherapy technology. When ray “strikes” solid tumors, it can irradiate tumor lesions vigorously while avoiding normal tissue irradiation, so as to maximize the curative effect. The broad spectrum of LES energy is focused into a beam, and the effect is concentrated. A small area can perform precise regulation and repair on tumors and some difficult diseases. The emphasis here is on gentle balance regulation, because only non-lethal effective control of viruses and bacteria will not produce these viruses and bacteria. The tragic consequences of struggling to survive are often incredible. It is completely different from the existing medical treatments, which are based on the thinking and methods of elimination. In this way, the medical field will replace the thinking and method of eliminating pathogenic factors with a gentle balance adjustment method. A truly natural natural therapy is emerging globally and tending to a wide range of medical and health fields.

LES contraceptives. Nano-level LES+Ag&Cu Zelile technology materials are added to condoms, which can help improve the blood and body fluid circulation of the sexual organs of both men and women, and help improve the quality of sexual life of both men and women, so that both men and women can Enjoy the unprecedented feeling. It can also inhibit the infection of viruses and bacteria, and I believe it will be welcomed by users.

LES can effectively control diabetic gangrene and even cure patients. LES can also help amputees heal without a second operation. This can save the lives of amputees caused by various reasons and reduce the trouble of amputees.

LES technology will help the growth of rare drugs. For example, with the help of LES smart technology, Ganoderma will grow significantly. More importantly, the spectral environment of Ganoderma lucidum can process spore powder of Ganoderma lucidum = a life-regulating substance with more life energy, which is considered to be beneficial to human health. It will more obviously help control tumors. According to the report, Ganoderma lucidum spore powder contains the following active ingredients, and the use of LES smart technology will promote the effects of these ingredients.

### Ganoderma Lucidum Polysaccharide

It can enhance the function of the immune system; lower blood pressure and prevent cardiovascular disease; accelerate blood microcirculation, improve blood oxygen supply capacity, and reduce the ineffective oxygen consumption of the human body at rest.

### Ganoderma Triterpenes

Ganoderma triterpene is an important pharmacological component of Ganoderma lucidum, and the triterpene component of Ganoderma lucidum plays an important role in anti-tumor. Triterpenes are the main functional components (spores) of Ganoderma lucidum, which have anti-inflammatory, analgesic, sedative, anti-aging, tumor cell inhibition and anti-hypoxia effects. Experiments have proved that Ganoderma triterpenes can quickly improve immunity. It promotes the proliferation of lymphocytes and improves the phagocytic ability and lethality of macrophages, NK cells and T cells.

Improve microcirculation, reduce cholesterol, avoid hardening of blood vessels; enhance liver, spleen and gastrointestinal functions, and improve digestive organs

### Natural Organic Germanium

It can enhance the body’s blood supply, promote blood metabolism, eliminate free radicals in the body, and prevent cell senescence; it can absorb electrons from cancer cells and reduce their potential, thereby inhibiting the degradation and spread of cancer cells.

Adenosine

Inhibit platelet aggregation and prevent thrombosis.

Trace element selenium

Trace element organic selenium: prevent cancer, relieve pain, prevent prostate disease, and use with vitamin C to prevent heart disease and enhance sexual function.

LES nano silver fiber can be used for LES + nano silver fiber antibacterial fabrics, filters, LES + Ag&Cu Zelile nano anti-fog filter, etc. Industrial filtration, functional textiles, medical dressings.

Fermentation tanks sprayed with LES technology functional materials in the fermentation tanks required by the wine and pharmaceutical industries, as well as methods to filter water and increase the biological activity contained in the water using the LES method, and fermentation tanks using LES technology materials (the applicant went A large winery visited and believed that LES fermentation technology can bring innovation and change to the wine industry).

Because the LES has an intelligent control function, the intelligent control of the LES can also be used in various chips and the above-mentioned graphene film structure, so the various functions and circuits produced by it can show intelligence, which is of great significance..

LES smart graphene technology will cover the fields of smart chemical energy, electronics, communications and services. The chemical business scope includes basic materials, batteries, information and electronic materials, materials and life sciences. Also participate

In the field of graphene preparation, it mainly focuses on chemical vapor deposition, redox, embedding and exfoliation. Among them, chemical vapor deposition mainly involves the doping modification, transfer and patterning of graphene films. Mainly related to energy storage (lithium ion batteries, solar energy, fuel cells, etc.); electronic equipment (LED and OLED, transparent conductive film, touch screen, liquid crystal display, sensors, cables), etc.; water treatment, air treatment; chemical industry (thermal interface materials) , Rubber and plastic), catalysis, electromagnetic shielding, etc.).

### LES Sleep Quilt and LES Bedding Series Products

LES special sleep bedding uses countless bio-spectrum microbeads, and the surface of the quilt is made of bio-spectrum technology materials. Therefore, the use of healthy LES energy can maintain the user’s physical and mental health for a long time. The environment for mutual communication is beneficial to physical and mental health, and Significantly improved sleep problems. Sleep is the main problem facing the world today. Many methods have been developed by the medical and scientific communities, but have not yet been resolved. The applicant’s LES healthy sleep quilt and LES healthy technology mattress can be based on LES, and can be used in combination with the Buddha stone that can control viruses and bacteria; it can also be LES + negative oxygen ion technology; healthy, antibacterial and airtight sleep quilt For families, hotels, hospitals, nursing homes, etc., especially for government agencies, company executives, research experts and other people who think too much.

LES can promote the healthy development and growth of sperm. In the United States, 12-15% of couples cannot have children, and one third of them are caused by men. Sperm need to stay in the body for 72 days to mature, and then wait for the opportunity to be ejected. If a man has not had sex or ejaculation for a long time, the vitality of mature sperm will decrease. Using LES technology can promote sperm count, vitality and health, making it easier for both men and women to become pregnant.

LES can help intelligently regulate the secretion of testosterone. Testosterone is a steroid hormone secreted by male testes or female ovaries. The adrenal glands can also secrete small amounts. It can maintain muscle strength and quality, maintain bone density and strength, and improve physical fitness.

The main pharmacological effects of testosterone are:
1. Promote and maintain male secondary sexual characteristics;
2. It can reduce the excretion of chlorine, sodium, phosphorus and potassium in men and women;
3. The growth of bones, skeletal muscle, hair and skin, and increase the production of red blood cells, promote the formation of blood vessels and darken the skin, large doses can inhibit the secretion of gonadotropins;
Fourth, it can antagonize the effects of estrogen on female breasts and endometrium.

Nowadays, many white-collar workers in the office often feel fatigue, listlessness, and lack of energy and energy. Generally, doctors believe that it is caused by abnormal kidney function. Now it is beginning to find that testosterone is abnormal. Regular treatment of these people to promote the growth of testosterone is not bad, but long-term treatment is needed. The use of LES to treat the testicles will stimulate the secretion of testosterone hormones. This fundamentally solves this problem. There is no doubt that men and women with abnormal testosterone can be perfectly perfected in nature without side effects.

LES contraceptives. Nano-level LES+Ag&Cu Zelile technology materials are added to condoms, which can help improve the blood and body fluid circulation of the sexual organs of both men and women, and help improve the quality of sexual life of both men and women, so that both men and women can Enjoy the unprecedented feeling. It can also inhibit the infection of viruses and bacteria, and I believe it will be welcomed by users.

LES can effectively control diabetic gangrene and even cure patients. LES can also help amputees heal without a second operation. This can save the lives of amputees caused by various reasons and reduce the trouble of amputees.

LES technology can be used to make LES refrigerators, storage cabinets, boxes, so that the water, vegetables, fruits, meat, food, etc. placed in them are affected by LES, and the life energy of LES is obtained, so the above-mentioned foods obviously increase human essentials. The beneficial ingredients and contribute to the health of users.

Fruits and vegetables processed by LES technology already have rich life energy. If these fruits and vegetables are added to the life energy water processed by LES technology and placed in a container containing LES, they will be broken down into liquid, and people will drink this liquid. It will play an important role in inhibiting diseases in the body and enhancing human immunity

LES originated from nature, and the micro-millimeter wide-spectrum life energy technology (BST) intelligently controlled by the applicant of the present invention is more easily accepted by organisms. It is not only consistent with the macroscopic biology, but even with the microscopic. The halo field and life spectrum are also very similar to natural LES, and different from other physical fields, it can be quickly transmitted to cells within one minute, and can charge cells, enhance the life energy of organisms, and find that abnormal cells can mobilize organisms The immune system suppresses them, helps cells suppress disease and improves survival.

LES refers to the life energy in nature. From the macro to the micro life energy spectrum field, the applicant’s discovery and invention make it easier and faster for living beings to transmit and receive, and have intelligent identification and adjustment functions. This technology has achieved the purpose of influencing and improving the life process of organisms.

The organisms referred to by LES are human beings, animals, plants, agricultural seeds, animal husbandry, and all organisms with intelligent regulation from the macro to micro life spectrum information, including tissues, cells, genes and proteins.

The impact of LES on living things refers to the fact that LES is similar to the life energy of living things. Among them, the life aura of the microscopic life spectrum also plays an important role that humans have not yet discovered. In addition, it can intelligently and quickly identify normal and abnormal normal cells, and mobilize the immune system to suppress abnormal cells, and realize the use of artificial means such as drugs when the organism is abnormal, without side effects, intelligently regulates, and repairs diseases. Solve the discomfort in the organism, and complete the pure natural biological method to affect and improve the living environment of the organism. It refers to the impact of LES on nature and organisms, each of which can use various combinations of big data and intelligence to obtain better results.

Even if the mentioned LES does not add special measures, it will have a significant impact on biology.

The combination of LES + Ag&Cu Zelile technology forms a healthy air filtration system that can inhibit viruses and bacteria.

The characteristics of the silver, copper, and zinc compounds of the metal ion-containing zeolite are that the active ions can be stably retained on the zeolite for a long time and release the activity slowly, so its antibacterial effect is stronger than its compound. It has good anti-virus, anti-bacterial, disinfection, anti-mildew, anti-corrosion and other functions, so its use is getting more and more attention. LES exchanges with silver, copper and zinc ions to form LES + Ag&Cu Zelile combined antibacterial filter layer or filter on the zeolite, which can purify the air and trap halogen in public spaces (including vehicles, ships, hospitals, hotels), residences, etc. People provide fresh, sterile and healthy air. LES + Ag&Cu Zelile isolation doors can also be installed in the lobby, indoors and on each floor.

The healthy drinking water produced by LES + Ag&Cu Zelile technology has antibacterial, cleansing effects and is rich in life particles. The applicant also found that it can inhibit purines, prevent and alleviate the symptoms of gout. It also has the functions of eliminating fatigue, improving blood circulation and nerve function, and moisturizing the skin, similar to hot spring water. It can help people drink abundant healthy living water at home, and always enjoy bathing similar to hot spring water, which greatly improves people’s health.

### Health Level

LES + Ag&Cu Zelile has stronger antibacterial effect than its compound and lasts longer. The LES + Ag&Cu Zelile ion exchange method makes an antibacterial coating on the zeolite, which is widely used for disinfection in the environment of hotels, hospitals, theaters, conference halls, cars, and ship cabins to prevent people from cross-infection. It can realize aseptic hotels, aseptic hospitals, aseptic parks, aseptic vehicles and cabins, etc.

The LES + Ag&Cu zeolite metal zeolite containing active ions can also be used for the preservation of fruits, vegetables, flowers, etc.

LES + Ag&Cu Zelile can also be made into a water container to keep the water clean and full of life energy. Keep things fresh, moisturizing and not perishable.

The naturalization of human space. The applicant proposes that the use of LES + Ag&Cu Zelile technology can change the long-term living space of humans and organisms into a natural biological environment including organisms, making it the same natural ecological environment as mountains, forests and rivers. This is also a human dream. LES + Ag&Cu Zelile technology can be used to simulate fresh air and natural gas fields in nature, whether it is walls, ground, roof or air. In this way, humans do not have to climb mountains and wading during the holidays, and breathe the fresh air of nature. Let nature be at home, on the way, office, sleep. People’s discomfort when traveling at high altitude may be greatly improved. Away from the sea, the distance between the sea and the land may not be so tiring. LES technology may be a gift to mankind.

LES + Ag&Cu Zelile can be used for wood, paint, decorations, air filtration systems and other building materials. LES + Ag&Cu Zelile can be used to simulate the reproduction of nature whether it is walls, ground, roof or air. In this way, humans do not have to climb mountains and wading during the holidays, and breathe the fresh air of nature. Let nature be at home, on the way, office, sleep. People’s discomfort when traveling at high altitude may be greatly improved. The long distance between the seabed, the sea and the land may not be so tiring anymore. Life energy spectrum technology may bring gifts to mankind.

LES + Ag&Cu Zelile forms a scanning filter field that can inhibit viruses and bacteria. LES + Ag&Cu Zelile technology can penetrate into wood through a variety of methods, so that the wood is rich in LES + Ag&Cu Zelile. Coatings and paints can also use LES + Ag&Cu Zelile technology to increase the life energy spectrum of coatings and paints. Various decorations have adopted technology to enrich LES + Ag&Cu Zelile decorations. The indoor ventilation system adds LES + Ag&Cu Zelile virus killing and bacterial filtration system, which can transform indoor air into healthy air rich in LES + Ag&Cu Zelile, which is essential for the spread of the new coronavirus in today’s world. The applicant designed the LES + Ag&Cu Zelile scanning and sterilization channel field technology suitable for various human and animal access channels. LES + Ag&Cu Zelile technology can penetrate into wood through a variety of methods, so that the wood is rich in LES + Ag&Cu Zelile technology. Coatings and paints can also use LES + Ag&Cu Zelile technology to increase the LES energy field and exert antibacterial and beneficial effects for a long time. Various decorations adopt LES + Ag&Cu Zelile technology, and enrich LES + Ag&Cu Zelile decorations. The indoor ventilation system adds LES + Ag&Cu Zelile virus killing and bacteria filtration system, which can transform indoor air into healthy air rich in LES + Ag&Cu Zelile. It is vital for the new coronavirus to sweep the world today. The LES + Ag&Cu Zelile scanning and sterilization channel field technology designed by the applicant is suitable for various human and animal channels. It can be realized on the way, in the office and in sleep, so that people who love nature can get rid of artificial isolation, such as reinforced concrete, and always breathe fresh air in the natural environment. People’s high altitude travel may be greatly improved due to extreme fatigue caused by the long distance from nature. In addition, LES + Ag&Cu Zelile seats and carpets will also have various bacteria and viruses brought by passengers from all over the world, and they will circulate in circulation. The space is fully controlled, bringing passengers a truly comfortable, healthy and sterile environment. Long distances to the seabed, ocean and land can also make you feel comfortable and healthy. Hospitals and hotels are disinfected to help people live in natural, fresh and healthy air and environment every day. Especially long-term deep diving crews will also use LES + Ag&Cu Zelile technology in the submarine. From the environment on board to the diet of the crew, they use LES + Ag&Cu Zelile technology to benefit the crew. It is very important for the military to control the deep changes caused by hypoxia and fatigue in the body, and effectively improve and maintain good mental and health conditions. LES technology may also be a gift to mankind in these aspects.

LES technology is combined with a variety of adsorption materials, such as photocatalyst + sepiolite, + attapulgite clay + negative oxygen ion + activated carbon, which has been called a new type of material for the entire century. These natural ingredients absorb viruses and bacteria. Formaldehyde, benzene, ammonia, TVOC paint binder, paint, various odors and other harmful gases, dust and other harmful ingredients. At the same time, LES can make the air cleaner and contain life factors, which is more beneficial to public health. The product has strong catalytic activity under ordinary light, ultraviolet light, especially LES broad-spectrum radiation, and can oxidize and decompose many toxic and harmful pollutants into non-toxic and harmless substances, such as CO₂, H₂O, and achieve better removal.

### Smelly Effect

Antibacterial, good for health. In addition, in the broad spectrum of LES, the filter can be automatically regenerated and can be reused for a long time. This expands the scope of application.

The product uses LES and metal foam nickel as carriers, with stable performance, large specific surface area and high catalytic efficiency. It is widely used in air conditioners, air purifiers, homes, offices, conference halls, hotels, hospitals and other fields.

The main planting technical parameters are as follows: to

Deodorization rate (%) Antibacterial killing rate (%) Thickness (mm) ≥98≥ 981.5-6.5

Under the broad spectrum of LES, the photocatalyst can decompose formaldehyde and organic matter by more than 99%, and because the process adopts the principle of contact catalytic oxidation, the catalyst component acts as a medium, so the photocatalyst component is almost unchanged, So it can be used for a long time without any treatment.

Practical function-it can be cleaned, the effect will not deteriorate after cleaning.

The basic material used in the photocatalyst filter is: including LES technology + foamed metal nickel and other basic materials

Scope of application: air conditioners, filters and other places where filters can be used.

Features: Compared with the traditional aluminum-based honeycomb network, the effective contact area is greatly improved, and the ability to receive the LES spectrum (including ultraviolet) is also greatly improved.

The coarse-pore sponge filter adopts LES technology + ordinary open-pore soft foam through net treatment. The mesh process removes the original mask or wall film between the foam networks and obtains the main skeleton network structure. The porosity of the foam is as high as 97%, and it has excellent air permeability, good flexibility and high mechanical strength. Using mesh foam as a filter material has the advantages of low flow resistance and high filtration rate. It can effectively filter out particles with a particle size of 15UM. LES technology + mesh foam has many special uses, such as filter materials, fuel tank filling materials and fabric processing materials. With reticulated foam as the framework, many new LES technical functional materials can be produced, such as LES grid ceramics, LES grid metal and LES grid activated carbon, which are good for health, deodorization, killing viruses, bacteria and purification air. In addition, the mesh bubbles are easy to clean and can be reused. It is a multifunctional and economical filter material that can suppress odors, eliminate viruses and bacteria, purify the air and be beneficial to health. Can be widely used in industrial production, vehicles and ships, aviation and civil equipment filter devices. At the same time, LES filters with excellent flame retardant effects can be manufactured according to customer requirements.

The applicant found that the LES + Ag&Cu Zelile technology can enrich the water with life energy particles. The function of LES + Ag&Cu Zelile water is to remove odors and remove heavy metals (the commonly used water is hard water, and our LES + Ag&Cu Zelile water is soft water). The difference between hard water and soft water is: drinking hard water is discharged from the stomach to the kidneys. And there are many impurities remaining, these impurities will affect the normal function of the stomach to the kidneys and cause various diseases. LES + Ag&Cu Zelile soft water is absorbed by cells immediately after drinking, can participate in systemic metabolism well, and obviously control purine. Suitable for human daily drinking and bathing. Suitable for the health of humans and organisms. It can not only promote cell absorption and growth, but also has a similar effect to hot spring water, smoothing and cleaning fat, and relaxing the body and mind.

Through quantum entanglement, the possibility of information transmission between particles was discovered and understood. This is very important for the energy transfer of LES.

The applicant found that with the advancement of technology, people’s physique is deteriorating. In terms of human body temperature, in the 1960s and 70s, people’s normal body temperature was 37 degrees, and now people’s body temperature is 36 degrees, which is almost exactly 1 degree. How important this level is to us humans has caused many viruses and bacteria to take advantage of this defect, and how many people still get sick or even die due to lack of this level. LES intelligently raises people’s basal body temperature through endogenous thermal effects and non-thermal effects, thereby improving health and extending life. When a person is unwell or sick, the energy in the body will be weak. The most important thing is to accurately use the LES energy to recharge in time.

In addition to accelerating wound healing alone, LES + Ag&Cu Zelile can also be used together with commercially available zipper wound patches to facilitate and effectively treat wound healing. LES + Ag&Cu Zelile can stimulate heat shock proteins at the molecular level, thereby participating in the entire process of intelligent wound healing. Mobilize platelets, activate 8 factors, give full play to the role of hemostatic factors, form a fibrin clot near the wound, make the wound have hemostatic effect (LES) quickly (within about 10 minutes), and quickly (within 30 minutes) all including cancer All kinds of pain including pain have analgesic effect. Wound healing or wound healing is the natural process of human skin and epidermal tissue regeneration after trauma. Usually, the epidermis (outermost layer) and dermis (inner or deep layer) of the skin are in equilibrium to form a barrier to protect the wound. When the char ruptures, the normal wound healing process will begin quickly. The basic process includes the following stages: hemostasis, inflammation, proliferation, maturation and remodeling. When the skin is damaged, (LES) will help the body produce complex biochemical processes, which will help quickly repair various wounds.

The LES + Ag&Cu Zelile technology is used in conjunction with its own zipper wound tape to accelerate wound healing and prevent infection. (See FIG. 5)

A. The day of surgical suture. B. LES posted for 6 days.

(The zipper wound patch with LESAg&CuZelile technology can prevent wound infection and accelerate wound healing.)

LES + UV = full spectrum light source. It is used to promote the development and growth of organisms, and inhibit viruses and bacteria. In these respects, it is much better than a simple light source.

Since LES is at the molecular level, it uses cells, genes, proteins and other life particles to control diseases and maintain the balance of the life cycle, especially cancer pain (a medical problem). LES should also have a significant inhibitory effect. An applicant has brain lymphoma that has metastasized to the liver and also has advanced liver cancer. The degree of liver pain is more painful than the first ten indicators prescribed in medicine, and painkillers can only be taken for 3 hours at a time. The applicant uses LES for pain relief. Unexpectedly, the episode’s unbearable late liver pain disappeared 30 minutes ago. After doing this a few more times, the liver pain will bid farewell to the applicant. The oncologists at New York Presbyterian Hospital find it incredible that the king of tumors, the pain of advanced liver cancer is so simple that it can be solved without any medication. This is also the applicant’s desire to obtain approval and support from the Patent Office as soon as possible, so that patients with various pains all over the United States and around the world can quickly and simply control the pain and treat the disease.

Applicants use LES technology to propose and develop private health banks. Sensors on the human body monitor various indicators in the human body 24 hours a day. Once any indication is found to be abnormal, the user and monitoring headquarters will immediately be prompted to participate in the LES. Effective control and maintenance measures.

The applicant also uses LES technology to monitor the user’s life intelligence index system around the clock. When a person falls asleep, the vital intelligence index is 100%. When a person wakes up, the cerebral cortex starts to move and consume energy. Wake up activities and further consume energy. Many users arrive at 10:11 in the morning and 3-4 in the afternoon. Users are prone to lack of energy and exhaustion, and it is difficult to keep working. Because LES regulates cells, genes and proteins, and repairs the root cause of fatigue, LES can eliminate fatigue and restore energetic health within 30 minutes, even for exhausted patients. status. For those who are prone to fatigue, this purely natural method of eliminating fatigue is also a great boon. Adding the botanical Perrin, the following effects are increased,

Herbal medicine Pelan has the following effects:

First, it can effectively alleviate the symptoms of fever and cold, headache, no sweating, body aches or pains, dizziness, swelling and pain caused by a cold.

Second, Perrin also has anti-inflammatory and sterilizing effects. It contains a lot of volatile oil and nerol acetate and other substances. These substances can effectively inhibit some typhoid bacillus, Staphylococcus aureus, and diphtheria bacillus. Bathing with Perrin can be effective Prevent a variety of skin diseases.

Third, Perrin can also treat viral influenza, because its volatile oil components can effectively inhibit some influenza viruses.

Fourth, the removal of bad breath is also one of the main effects of Peilan. For those bad breath caused by long-term bitterness and poor stomach, you can take 50 g of Peilan leaves, decoct them in water or soak them as tea for a period of time. Can effectively improve the problem of bad breath.

Fifth, Peilan also has the effect of refreshing and refreshing. Peilan has the effect of helping resuscitation and refreshing, and relieves symptoms such as neuropathic headaches and cold headaches.

Sixth, Perrin can also relieve acute gastroenteritis.

Promoting body fluid to quench thirst, regulating qi and blood, diuresis and regulating phlegm, etc. In LES functional spray, functional mouthwash, and functional toothpaste, plants and compounds that have been discovered and proven by the East and the West can be reasonably added or subtracted in the formula according to the needs of different users to meet the needs of different users.

LES spray, LES mouthwash, and LES functional toothpaste solve the limitations of traditional mouthwashes, traditional toothpastes that do not sterilize, remove scale, prevent bad breath, and cannot inhibit Helicobacter pylori, while promoting the repair of gum damage. In these functional sprays, mouthwashes, and functional toothpastes, the traditional Chinese medicine Panax notoginseng (to promote blood circulation and remove blood stasis, help to repair gum bleeding and damage), add anti-calculus and inflammation-removing bacteriostatic peptides, seabuckthorn liquid, and add can Remove yellow tartar, add vitamin E, D to clean and repair oral inflammation, add ginseng, wolfberry, can help the health of the mouth and stomach.

LES fracture accelerated healing support series. In the past, plasters or braces were often used for fractures, which allowed the callus at the fracture site to recover naturally and slowly, which took a long time and was painful for the patient. Adding LES technology to the support can improve fluid circulation, repair the nervous system, and accelerate the formation of callus. Generally, the callus at the fracture site can be formed in about two to four weeks, which significantly shortens the healing process..

The LES functional patch can be used in the treatment and rehabilitation of various inflammations, wounds, and diseases. It can inhibit virus and germ infections and accelerate the healing of diseases.

LES anti-virus spreading masks, add LES + Ag&Cu Zelile or + rare earth element germanium-based negative oxygen ion comprehensive technology in the raw material of the mask to achieve the effect of anti-virus and promote health.

In the bedding, toilet, bathroom surface and supplies in hospitals and hotels, especially the cleaning pads often replaced in hospital beds, LES + Ag&Cu Zelile or + rare earth element germanium-based negative oxygen ion comprehensive technology can be added to inhibit Virus infection, inhibit bacterial infection, and benefit the health and comfort of users. It is a must to become a sterile hospital and a sterile hotel.

Light source + LES = the new light source of LES life energy. No matter where this light source emits light, it has obvious health-promoting effects on life systems. LES can be added to various light sources to form new light sources rich in LES energy fields. The applicant believes that in some countries, LES can also be added to the artificial sun under study to produce an artificial LES sun. If LES is added to an artificial satellite, an artificial moonlight rich in LES can be created, and it can be used more widely. Let the LES artificial light source illuminate more people, and let the LES energy field bring them health information rich in life energy, so as to benefit the public.

LES + Ag&Cu Zelile heat insulation and sunscreen, can photodegrade formaldehyde, inhibit viruses and bacteria in a wide range, enhance indoor life, health and fresh air, and protect privacy. Can be used indoors, offices, halls, vehicles and ships

There are also sun rooms and so on.

The LESAg&CuZelile intelligent control system can also be applied to the entire biotechnology system, such as agriculture, from seed development, growth to fruit, including intelligent control of the nutrient content of various agricultural varieties and the nutrients required by humans. It will greatly increase unnecessary substances Or harmful substances to humans will be greatly reduced, which will help discover and cultivate new agricultural species. It can also greatly increase the nutrient content of various crops and fruits, thereby helping to improve the public’s immunity and health.

Using LES antibacterial technology in mobile phones can inhibit harmful microorganisms such as viruses and bacteria. LES technology can be installed in mobile phone panels, casings and panels made of plastic, ceramic and various materials.

The aircraft uses LES to simulate the natural environment, fills the aircraft with natural life energy, controls bacteria and viruses, so that people are full of vitality in the bio-energy environment full of natural environment, and realize physical and mental comfort.

LES is used in high-speed railways and trains to simulate the life energy in nature and control bacteria and viruses, so that people can be energetic in the bioenergy environment full of natural energy and get a comfortable environment in mind and body.

The LES on the ship uses bio-energy that mimics the natural world and controls bacteria and viruses, so people can be full of vitality in the natural environment of bio-energy and realize physical and mental comfort.

The submarine uses LES to simulate the bio-energy technology in nature, which can reduce the fatigue of the crew and complete the important task of submarine diving full of energy.

The car uses bio-energy technology to control bacteria and viruses, so that people are full of vitality in the bio-energy environment in the natural environment and realize physical and mental comfort.

LES functional facial mask LES functional beauty spray and LES functional cosmetics series can improve the skin condition of the face from the molecular level, so that users can continue to maintain the luster, elasticity, firmness and rosyness of the facial skin. This may be difficult for other facial masks and cosmetics. Did it. If you use the LES beauty skin care instrument at the same time, the effect will be better. LES functional facial mask, LES functional beauty spray and LES functional cosmetics, coupled with LES beauty and skin care instrument, can also remove all kinds of scars, acne, etc., helping users maintain a whitening, moisturizing, firming, and youthful charm for a long time face.

### LES Furniture Spray Functional Film

On the basis of the existing furniture functional spray film, LES+Ag&Cu Zelile technology is added to form a functional spray film that inhibits viruses and germs and promotes health, which is beneficial to beautify furniture and purify indoor air.

LES functional film for heat insulation and shading doors and windows

LES technology can increase the function of sunscreen and heat insulation, UV protection, formaldehyde prevention, help control viruses, germs, radiation to the indoor air in the indoor space to promote healthy life particles, and one-way perspective during the day.

The LES+Ag&Cu Zelile technology is added to the existing doors and windows for heat insulation, light protection and privacy, which increases the inhibition of viruses and germs, and helps the indoor environment, air purification and health.

Agriculture, forestry, horticulture improve varieties, create new varieties, implement LESAg&CuZelile intelligent supervision from the inside, realize drought resistance, flood resistance, insect resistance, increase yield, increase human nutrition and taste, and help improve public health on a large scale, immunity.

LESAg&CuZelile implements large-scale forestry development, prevents various pests and diseases, and cultivates new varieties.

LESAg&CuZelile has improved and developed horticulture and flower varieties, prolonged the flowering period, and made the flowers more beautiful and colorful.

LESAg&CuZelile protects and develops rare varieties.

Animal husbandry, bird improvement and development of new breeds, especially the use of LES technology to extract eggs from ovaries from high-quality production areas and fertilize them into high-quality embryos. Then go to the barren places to give birth to local animals and animal husbandry through the abdomen, obtain purebred high-quality breeds, which can eliminate diseases such as mad cow disease and achieve large-scale high-quality development worldwide. Including the development of natural food and the control of pests and diseases. Maintain and develop rare species,

LESAg&CuZelile is used to improve and innovate the types of fish and various marine life to enhance immunity and resist various storms. Improve the taste and ornamental value.

In waters lacking fish and marine life, LESAg&CuZelile technology is used to reproduce and reproduce marine life (such as corals, etc.) and fish, and save the ocean and water environment of humans.

Food development is a natural source of nutrition for food and beverages. Using LESAg&CuZelile technology, starting from the water and fermentation environment, making full use of the rich living water, adding LESAg&CuZelile technology to the fermentation raw materials, covering the stainless steel fermentation tank cavity on the LESAg&CuZelile technology layer, comprehensive reform and development, and obtaining high quality throughout the process The speed of wine will be greatly increased. Using LESAg&CuZelile fermentation technology can reform many industries, such as the pharmaceutical, wine and food industries.

Improve the cultivation of LESAg&CuZelile natural medicinal materials and develop the varieties of natural medicinal materials. The whole process of seed growth and results is affected by the life energy spectrum technology, including water, soil, air and light. Good medicine can be made and taken.

Using LES+Ag&Cu Zelile technology to treat various wastewater and waste can help convert wastewater into LESAg&CuZelile-rich reclaimed water. Except for bricks, stones, steel, glass, etc., most wastes can be fermented through the life energy spectrum technology. , Become a good feed and fertilizer. Contribute to the development of wastewater treatment, biological purification and bioenergy.

LES + Ag&Cu Zelile technology smart film is mainly used in smart film greenhouses for growing crops. The intelligent effect of LES + Ag&Cu Zelile film is: 2 to 3 degrees higher than the general film temperature, with insecticides, no pesticides, no water droplets, drought and flood resistance. Under this year’s floods, most crops have been reduced in output and the ability to produce fruits has basically been lost. At the experimental site of LES, the rice has lodging resistance, the rice is full, and a good harvest is expected. It matures 2 to 3 days earlier than normal membranes. For farmers, progress is benefit. LES + Ag&Cu Zelile saves farmers a lot of money. The cost of pesticides is equal to the labor cost of pesticides. After trial, it was favored by farmers.

LES + Ag&Cu Zelile soil total nutrient improvement system method. Nowadays, in many countries, the land has been cultivated for a long time, only harvesting but no nutrients, causing the land to become more and more scarce, and the nutrients needed by many human bodies become less and less, and some tend to zero. Therefore, people’s health is now generally declining. Improving the soil and adding supplementary nutrients has become a top priority. Soil improvement is not just a matter of adding fertilizer. It must be scientifically improved. First measure the nutrient deficiency in the soil in various places, and then aim at the nutrient deficiency, use the LES + Ag&Cu Zelile technology to specifically cultivate the nutrients rich in the required nutrients. The use of organic fertilizers that are grown with vegetables, fruits and grains and are rich in nutrients. These soils are cultivated and matured and integrated with these soils, which will greatly benefit the health of the people.

This kind of intelligent control system can also be applied to animal husbandry, and it will also bring huge changes to the development of animal husbandry.

This kind of intelligent control system can also be applied to fishery and marine life,

Similarly, it will also play an important role in the cultivation and development of animals, animal husbandry, poultry and birds, and the protection and development of rare animals and species.

The applicant found that LES can help understand biochemical and organic chemical reactions at the molecular level. In the late 1980s, Canadian scientist Gedye et al. made certain chemical reactions in a special PTFE sealed container greatly increased in a short period of time, but it was difficult to do in ordinary environments. However, the spectral electromagnetic waves used by LES are far below the prescribed electromagnetic radiation safety standards, but it will shorten and speed up the biochemical and organic chemical reactions. This is because of LES! Broad-spectrum electromagnetic waves including micro-millimeter waves, whether polar solutions or non-polar solutions used for biochemical reactions or organic chemical reactions, absorb micro-millimeter wave energy from the molecular level according to the polarity and molecular pairs of molecules. As the polarity of the molecule changes or the dipole increases, electromagnetic waves also increase. The greater the polarity of the molecule, the greater the energy absorbed by radiation, and the absorbed energy will cause the transition of vibration and rotation energy levels, which is the “internal heat effect” proposed by the applicant. Therefore, LES has a spectrum similar to biochemistry and organic chemistry at the molecular level, and has intelligent regulation. Research experiments in biochemistry and organic chemistry laboratories play an important role. It will also bring about new methodologies formed by biological and organic chemical reactions and changes in the chemical industry.

The use of LES technology in textile fibers, such as adding nano-level technology in stretching, spinning and blending, or spraying coatings containing LES on fabrics, can help keep warm, dry and dry in severe cold and expeditionary environments. Inhibit bacteria and improve blood circulation. It can be used to add LES technology to people’s clothing and wearable devices, and it can be equipped with a tracking generator in the LES device so that the user’s location can be found in time when needed.

The intelligent control system can also be applied to the entire biotechnology system, such as agriculture, from the development and growth of seeds to the results, including intelligent control of the nutrients of various agricultural varieties, which will affect the nutrients needed by humans. Significant increase, the unnecessary or harmful substances to humans will be greatly reduced, which will help discover and cultivate new agricultural varieties. It can also greatly increase the nutrient content of various crops and fruits, thereby helping to improve the public’s immunity.

### Ability and Fitness Level

This kind of intelligent control system can also be applied to animal husbandry, and it will also bring huge changes to the development of animal husbandry.

This kind of intelligent control system can also be used for breeding and developing fishery and marine life,

LES + Ag&Cu Zelile combines with germanium in rare earth to produce negative oxygen ions, which are beneficial to the environment and health. Life sciences have clarified that the main cause of disease and aging is related to oxidation. The substance that prevents oxidation is a reducing agent. For life sciences, antioxidants are healthy materials. Acidic substances that are essential to humans, such as food, meat, sugar and food additives, are actually harmful oxides sometimes. Excessive fatigue is also the cause of oxidation. Cations accelerate the oxidation reaction, and the polluted air contains a lot of cations, which makes people feel very uncomfortable. LES + Ag&Cu Zelile + germanium-based rare earths can intelligently suppress positive ions and generate negative ions, making people feel comfortable and beneficial to health.

Combining LES + Ag&Cu Zelile with germanium in rare earths can also solve the deacidification effect of meat, grains, sugar, food additives and other acidic substances. Eating non-acidified food will also become one of the necessities in people’s lives.

Negative oxygen ions play an important role in the physiological functions of the human body. When the negative ion concentration is greater than 1000/cm3, it is beneficial to human health and longevity, because it can improve sleep, lower blood pressure, promote metabolism, improve immunity, reduce fatigue, improve work efficiency, clean the air and other beneficial effects.

Germanium-based LES + Ag&Cu Zelile + rare earth technology has formed an emission material that can release negative ions, which is very beneficial to human health. It can be used in many fields such as construction, environmental protection, textiles, etc., to provide a way for people’s daily life. Practical, brand new, multifaceted healthcare. At present, general anion materials are synthetically synthesized LES + Ag&Cu Zelile + germanium-based rare elements through artificial synthesis of LES + Ag&Cu Zelile + germanium-based rare earth elements. The anion generating material is directly added to ceramics to make anionic ceramic materials.

### Earth Element Technology

Naturally inhibits bacteria and regulates life systems. There are 32 electrons around the germanium nucleus, and the 4 electrons on the outermost orbital move irregularly. Under the influence of LES’s internal heat effect + thermal effect, once the temperature rises, the electrons on the outermost orbital will deviate due to stimulation. Under the control of LES, the off-orbit electrons help to adjust the ion balance of the organism, restore the abnormality of the human body’s neural circuit to normal, prevent and improve the body’s discomfort and massage the effect of hot springs. The combination of LES and rare earth germanium also has the function of regulating the abnormal potential of the human body. When the potential of cancer cells rises sharply, the combination of LES + Ag&Cu Zelile + germanium-based rare earth element technology will control virus infection at the molecular level and control the electrons of cancer cells. Make its potential drop, inhibit the deterioration of the disease. The applicant’s control of viral infectious diseases, AIDS and certain tumors shows that this can be expected. LES will facilitate the development of communication between humans and nature, and coordinate research and applications. LES + anion generating materials that integrate multiple energies can not only reduce the production cost of anionic ceramic materials, but also greatly promote the large-scale application of LES + anion functional technology materials that integrate multiple energies.

LES + Ag&Cu Zelile technology is used in mobile phones, tablets, computers, glasses, toys, etc., which can significantly inhibit harmful substances and microorganisms, such as viruses and bacteria, and help the public use these daily necessities in a clean and safe manner. It can also be made into disinfectant, film, spray, paint, used in hotels, hospitals, offices, theaters, conference halls, residences and other places where people often touch and disinfect.

The comprehensive technology of LES + Ag&Cu Zelile + and rare earth element germanium-based negative oxygen ion has a wide range of uses. The applicant initially listed some of the following uses, and the rest will be submitted in the formal application.

LES + Ag&Cu Zelile + and rare earth element germanium-based negative oxygen ion comprehensive technology can be widely used in medicine, rehabilitation, health care and longevity. LES + Ag&Cu Zelile + and rare earth elements germanium-based negative oxygen ion comprehensive technology agriculture, LES + Ag&Cu Zelile + and rare earth elements germanium-based negative oxygen ion comprehensive technology, livestock, animal and poultry industries. LES + Ag&Cu Zelile + and rare earth element germanium negative oxygen ion comprehensive technology life sciences, LES + Ag&Cu Zelile + and rare earth element germanium negative oxygen ion comprehensive technology clothing industry, LES + Ag&Cu Zelile + and rare earth element germanium negative ion oxygen ion Comprehensive technical building materials, LES + Ag&Cu Zelile + and rare earth element germanium-based negative oxygen ion comprehensive technology decorative materials, LES + Ag&Cu Zelile + and rare earth element germanium-based negative oxygen ion comprehensive technology food industry, LES + Ag&Cu Zelile + and rare earth element germanium-based Negative oxygen ion comprehensive technology beverage industry, LES+Ag&Cu Zelile+ and rare earth element germanium-based negative oxygen ion comprehensive technology water industry, LES+Ag&Cu Zelile+ and rare earth element germanium-based negative oxygen ion comprehensive technology, fermentation technology, LES+Ag&Cu Zelile+ and Rare ear element germanium-based negative oxygen ion comprehensive technology brewing industry, LES + Ag&Cu Zelile + and rare earth element germanium-based negative oxygen ion comprehensive technology reaction technology, LES + Ag&Cu Zelile + and rare earth element germanium-based negative oxygen ion comprehensive technology, mushroom cultivation technology, LES + Ag&Cu Zelile + and rare earth element germanium-based negative oxygen ion comprehensive technology indoor health care technology, LES + Ag&Cu Zelile + and rare earth element germanium-based negative oxygen ion comprehensive technology can be used in clothing and footwear to improve the surrounding circulation, fitness, Deodorization and fatigue, as well as the cold and warmth of skiing, health care equipment, LES + Ag&Cu Zelile + and rare earth element germanium-based negative oxygen ion integrated technology strain fermentation technology, LES + Ag&Cu Zelile + and rare earth element germanium-based negative oxygen ion integrated technology t Source, LES+Ag&Cu Zelile+ and rare earth element germanium-based negative oxygen ion comprehensive technology air purification system, LES+Ag&Cu Zelile+ and rare earth element germanium-based negative oxygen ion comprehensive technology air purification system, LES+Ag&Cu Zelile+ and Rare earth LES + Ag&Cu Zelile + and rare earth element germanium-based negative oxygen ion comprehensive technology health club, LES + Ag&Cu Zelile + and rare earth element germanium-based negative oxygen ion comprehensive technology health leisure home, rehabilitation and elderly care center, LES + Ag&Cu Zelile + and Rare earth element germanium-based negative oxygen ion comprehensive technology health chain center, LES + Ag&Cu Zelile + and rare earth germanium-based negative oxygen ion comprehensive technology hot spring, LES + Ag&Cu Zelile + rare earth element negative oxygen ion comprehensive technology, sand bath germanium, LES + Ag&Cu Zelile+ and based on rare earth The negative oxygen ion sub-integration technology of element germanium wine industry, LES + Ag&Cu Zelile + and rare earth element germanium-based negative oxygen ion comprehensive technology fermentation technology, LES + Ag&Cu Zelile + and rare earth element germanium-based negative oxygen ion comprehensive technology biochemical reaction system, LES + Ag&Cu Zelile + and rare earth element germanium-based negative oxygen ion comprehensive technology water treatment system, LES + Ag&Cu Zelile + and rare earth element germanium-based negative oxygen ion comprehensive technology wide range LES + Ag&Cu Zelile + light source system and rare earth element germanium-based negative oxygen ion Comprehensive technology + ultraviolet light = comprehensive bio-energy light (used in organisms and places that need sunlight, such as the life energy spectrum light source of the soil, it should have a better sterilization effect than simple sunlight). LES + Ag&Cu Zelile + and rare earth element germanium negative oxygen ion integrated technology automotive lining system, LES + Ag&Cu Zelile + and rare earth element germanium based negative oxygen ion integrated technology aircraft application system, LES + Ag&Cu Zelile + and rare earth element germanium-based negative oxygen Ion integrated technology for vehicles, ships, ships application systems, LES + Ag&Cu Zelile + and rare earth element germanium-based negative oxygen ion integrated technology incubation system, LES + Ag&Cu Zelile + and rare earth element germanium-based negative oxygen ion integrated technology marine biological culture system, LES + Ag&Cu Zelile + and rare earth element germanium are the main negative oxygen ion integrated technology cooking technology system, LES + Ag&Cu Zelile + and rare earth element based negative oxygen ion integrated technology coating technology system. Under the effect of LES integrated technology, LES + Ag&Cu Zelile + and rare earth element germanium-based negative oxygen ion integrated technology roasting, mature coffee, tea (mainly Chinese Yunnan Pu tea) and other food and beverages that people drink and taste daily. Thousands of years of traditional crops have undergone fundamental changes, solving some of the ingredients that may hinder health in the traditional baking, fermentation and maturation process, so that people can truly taste the original natural aroma and deliciousness.

LES wild rare mushrooms, fungus cultivation technology, wild rare mushrooms can usually only grow in natural environments, such as specific air, humidity, temperature, etc., and it is not easy for people to find collectibles on the mountain, because people need to pay for expensive mountain treasures These treasures can only be tasted at a certain price. Fungi are mainly used in the pharmaceutical industry and laboratories, but they are also difficult to obtain and cultivate. The applicant believes that LES comes from nature. The development and growth of truffles, matsutake, dried mushrooms, morels and boletus also rely on the energy and information of LES to support the entire growth process. Therefore, it has been discovered that these rare wild mushrooms can be grown on a large scale throughout the alpine forest and even in planting bases with similar growth conditions, so that people can eat them economically and conveniently at any time.

LES researchers communicated with some people with advanced Chinese Qigong skills and found that some of them can measure part of the energy information of LES when they are in a good energy state, but they are unstable and long-lasting, usually when he thinks their skills enough. There is no information on the instrument. Some qigong practitioners often take a long time to practice qi, but before the LES device, they often feel qi within 5-10 minutes. This also indirectly illustrates the correlation between LES and people. Even the shoes, socks or clothes worn by some qigong practitioners contain LES technology, which may cause asthma.

Considering the relationship between sky, man and earth, spirit, spirit and cosmic air, the applicant also specially designed a special sole. Near Yongquan Cave, LES technology is adopted, which is breathable and waterproof, and is covered by existing common materials. It is surrounded by special materials so that when people touch the ground, the LES energy information from the sky and the earth can reach the ground from head to toe, penetrate the entire human body, and then encourage people to step on the uncovered land, and the human body can restore nature. The health of the wilderness. In addition, the sole channel for air conditioning and ventilation was designed and a pair of one-way valves were installed. Every step will exhaust the dirty air in the shoes. Because it is a one-way valve, air and moisture can only be emitted, and rain outside can not enter, which also keeps the inside of the shoe clean and dry.

You can also make all kinds of clothes, whether it is warm in winter or cool in summer, and can also be equipped with an intelligent monitoring system to know the user’s health information and location at any time, which is convenient for adventure, children and children.

### Old Man

Combining LES + Ag&Cu Zelile with rare earth germanium, and adding heat insulation and shading film on doors and windows, can fill the entire room with clean, sterile and healthy air.

The disinfection source made of LES + Ag&Cu Zelile + and the rare earth element germanium can be placed in the air to purify the air and kill viruses and viruses attached to the surface of various objects in the air on a large scale. This is very useful for families, conference rooms and auditoriums. Theaters, hospitals, hotels, clubs, etc. are very important. You can also use LES + Ag&Cu Zelile + and rare earth element germanium to perform various sprays and liquid sprays on indoor spaces and various objects to achieve the effect of cleaning and disinfection.

The applicant used LES technology to improve the nutrient content of peanuts, showing that LES can bring significant changes to all organisms, and nutrient content is one of them. Peanut (LES) technology processing peanut nutrient composition analysis results. The nutrient content of the treatment group is higher than that of the control group, indicating that (LES) technology can promote the growth and metabolism of peanuts, and the accumulation and storage of nutrients. Lysine, lysine, leucine, threonine and other essential acids belonging to the human and animal bodies, these amino acids are not synthesized by the human body, so they are all ingested from food, so (LES) processing can be Increasing the total amount of amino acids in food The peanut that must be used in the peanut industry has important nutritional significance. Minerals are also essential in the human body. Their functions include regulating body fluids to form a supporting framework and regulating chemical reactions in the body. In the processed peanuts, the total amount of the eight mineral elements measured increased. The role of these elements is: calcium, magnesium, etc. and phosphorus together form the skeletal system to support the body’s calcium and participate in blood coagulation, nerve tissue regulation, and participate in the control of heart activity and muscle activity. Potassium: has an effect on intracellular metabolism, can regulate the absorption of cardiac smooth muscle, and affect the excitability of nerve and muscle tissue. Sodium: When involved in body fluid regulation, muscle and nerve activity, sodium enters the cell, causing compensatory changes in the balance of cell membrane permeability. Magnesium: It is necessary to regulate the function of soft tissue, bone and nerve tissue through normal Ca/mg balance. It is also the basic activator of phosphotransferase. Iron: The main role is to form hemoglobin, which is responsible for oxygen transport and cellular respiration. Copper: It is a component of tyrosinase (polyphenol oxidase) and other enzymes. It is related to iron. Adding copper to the feed can obviously promote animal growth. Manganese: is a catalyst in metabolic activities, such as active zinc that constitutes phosphatase and arginase: it is a component of carbon dehydratase and alkaline phosphatase. Vitamin E, also known as tocopherol, plays an important role in the reproductive system. It is also an antioxidant in the body. When absorbed by the body, it enters the body at the same time as food fat and may accumulate in the body.

The applicant found that the yield of rice, wheat, watermelon and vegetables can be significantly improved, and the nutrient content is also significantly improved, and the taste is more memorable. LES technology significantly improves the nutrition and taste of seeds, seedlings or harvested products.

LES seed printing technology aims to solve the planting and cultivation problems of various crops and plant seeds. Using plant fiber or waste paper pulp that is easy to mix into the soil, adding LES + Ag&Cu Zelile + and rare earth element germanium-based negative oxygen ion comprehensive technology, planting all-element fertilizers, and then evenly distributing the seeds, and then printing the LES seeding board can be made into various Seed sowing boards for crops, plants, flowers and turf are easy to intelligently, large-scale planting and cultivation.

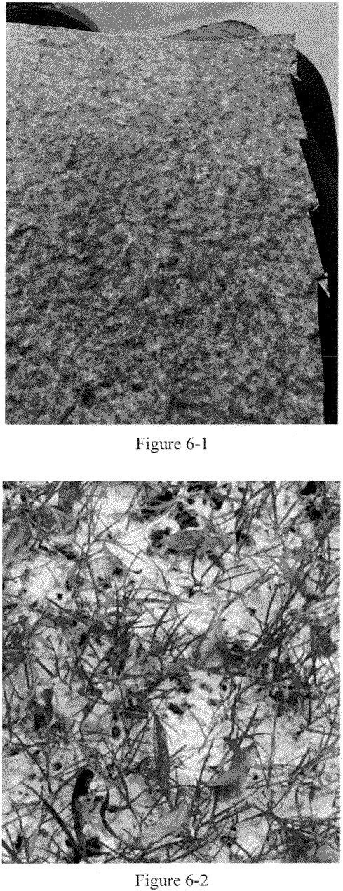

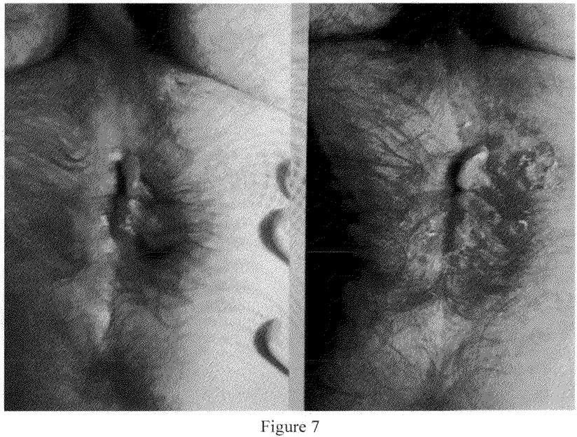

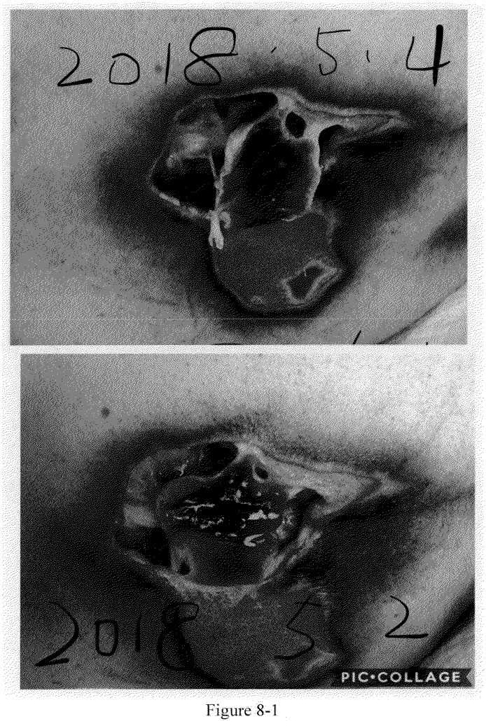

FIGS. 6-1 shows a schematic diagram of LES + Ag&Cu Zelile + and rare earth element germanium-based negative oxygen ion integrated technology seed cultivation board. FIGS. 6-2 is a photo of grass without LES treament.

LES originated in nature and the universe. Everything in the world is connected. The earth is a very small object in the universe, but the universe and the earth are also connected. I believe that to discover this inner connection, humans may have one. The way of communication from nature and from the universe is meaningful. Due to the influence of the atmosphere on electromagnetic waves, the electromagnetic radiation of celestial bodies has a very wide frequency, covering the entire electromagnetic spectrum from rays to radio waves (the upper and lower limits of the electromagnetic spectrum have not yet been determined). For the radiation waves of celestial bodies, the earth’s atmosphere has a characteristic: that is, only electromagnetic waves in certain spectrum bands are allowed to pass; while electromagnetic waves in other spectrum bands are either absorbed and reflected, or rarely pass. This characteristic of the earth’s atmosphere is called an electromagnetic radiation barrier. Generally, the electromagnetic spectrum that can pass through the earth’s atmosphere is called the “atmospheric window.” These windows are the remote sensing windows for the earth resources remote sensing satellite to observe the earth. The electromagnetic radiation outside the window is shielded by the atmosphere. The atmosphere is divided into three windows: visible light wave window, used for visible light remote sensing; infrared wave window, used for infrared remote sensing; microwave window, used for microwave remote sensing. LES comes from nature. In addition to assisting the connection with the earth, LES will also pass through the atmospheric barrier to establish a connection with the universe. Therefore, the applicant believes that it is necessary to seize the LES as an opportunity to study the new communication link between the earth and the universe.

The antibacterial ventilation of LES fans, such as Dyson or other fans, can add LES + Ag&CuZelile technology antibacterial filter layer and net on the wind blade or air outlet to make the air blown out fresh and healthy.

The materials of LES technology can reach the nanometer level. When making banknotes, bills and other important common papers, LES materials are added to inhibit bacteria and avoid mutual infection.

The applicant for the patent for the invention found that the invention originated in nature and applied modern technology, including improving the similarity and energy of the simulated life energy, so that the content published by the applicant for the patent for the invention can be helpful. Those skilled in the art without creativity. Through work, it is possible to realize the technical solutions applied by the present invention, and it is possible to master and understand the communication between humans and living beings, the exchanges and the amazing results of the impact and improvement on living beings.

### Implementation

The applicant of the present invention cited several test examples of the application of LES in medical, agricultural, animal husbandry and life science experiments.

Example 1. LES treats AIDS. The applicant found that LES can improve the immune function cell count CD4 of AIDS patients, and can cure herpes simplex, oral Candida infection, homosexual anal ulcers and skin ulcers in AIDS patients. The most incredible thing is that the director of the oncology department of New York Presbyterian Hospital diagnosed patients with multiple advanced malignancies. The patient’s immune function cell count CD4 only exceeded 30. It is believed that only AIDS can happen. After daily LES treatment, CD4 rose to 150 after half a year, and returned to normal after one year. Top medical experts believe that the patient cannot survive. He is still alive and is listed as a long-term follow-up case by NIH.

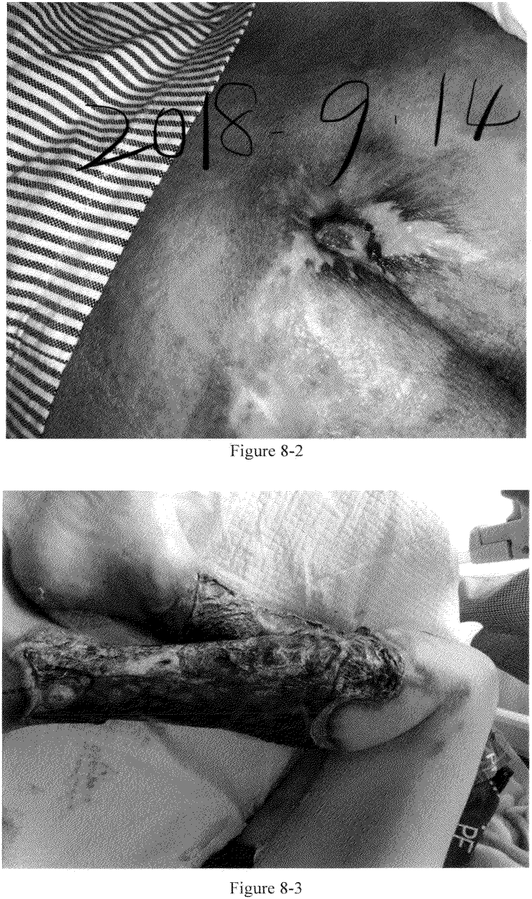

Comparison of LES treatment of AIDS and homosexual anal ulcers (see FIG. 7) .

Example 2. A patient undergoing liver transplantation was found to be swollen and weak shortly after the transplantation. The hospital found that he had kidney failure and the muscle liver was over 500. The doctor thought he would start dialysis and queue up for a kidney transplant. Results The patient was treated by a nephrologist and supplemented with LES. Unexpectedly, the edema disappeared after 1 month, the energy recovered, and the muscle liver dropped to 300. After half a year, the muscle liver even recovered to less than 100. This case also made the nephrologist feel incredible. So if the patent for this invention is to be approved, it will be of great significance to many patients with nephrotic syndrome and other diseases who are struggling on the edge of death in the United States and around the world, because they can get the assistance of nephrologists and LES The effective treatment of therapies may be cured without having to look for and wait for the transplantation of precious kidney organs. It is also possible that with the assistance of LES, it is possible to help those patients who live on renal dialysis to complete the self-repair function and end the kidney dialysis Dependence. This is also such a significant thing.

(The picture below shows the situation after liver transplantation.)

### Example 3

A person who was over 60 years old and had a malignant tumor. In March of this year, he might be infected when he went to the hospital and shopping mall two days ago. After coming back, he felt fatigue and fever (108° F.) in the morning after returning. The blood oxygen saturation was 94. It is more difficult. One side has a headache, lethargy, and boredom. The patient feels that all the symptoms are different from the viral colds and flus he had suffered in the past, and the symptoms seem to be more serious. At that time, it was at the peak of COVID-19 infection. Analysis may be suspected of COVID-19 infection, but I did not want to go to the hospital. Whether it was a cold or COVID-19 infection, I wanted to solve it at home. In view of the experience of LES curing the flu within 3 hours in the experiment, LES controlled the virus infection at the molecular level. The patient was treated in the LES environment immediately. After the whole body treatment for 30 minutes, the patient showed a layer of fine hair. Khan, took a shower, and drank 7 bottles of mineral water in one breath. At this time, the body temperature began to decrease, and the patient went to bed immediately. Then take body temperature and blood oxygen every 1 hour. The patient had a fever until 4 o′clock in the morning. At 4 o′clock, the body temperature was measured and it was found that the body temperature was almost normal, and the blood oxygen also rose to 96. At 8 o′clock in the morning, the body temperature had been normal, the blood oxygen had risen to 98, and all the symptoms of the patient had disappeared. However, the patient still felt weak and continued to sleep. At 2 o′clock in the afternoon, the patient took the initiative to get out of bed, feeling that he was almost normal. Feel like a normal healthy person. The applicant has worked with doctors in experiments to use LES in the early stages of colds and flu, and most of them are cured within 1-3 times. It deserves the attention of the Patent and Trademark Office. I hope that with your support, this natural remedy can be used as soon as possible to relieve the pain of more than 9 million COVID-19 patients across the United States.

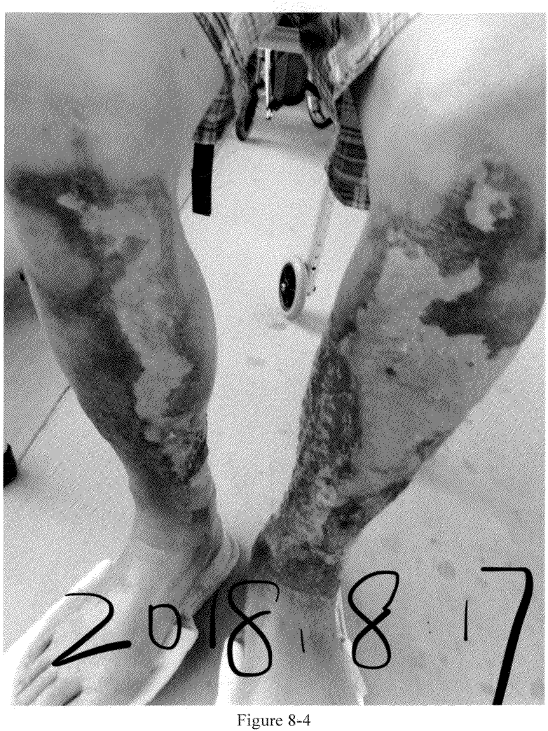

Example 4. A 90-year-old professor who took the wrong medicine caused striated muscle ulcers. The muscles and bones from the legs from the calf to the thigh were all separated. The ICU ward rescued and caused 4 degree bedsores and kidney failure in the buttocks. The hospital issued a critical illness notice, and the doctor thought it was impossible to treat. Relatives of the patient sought treatment from the applicant. The applicant analyzed the principle of LES and the patient’s disease status, and believed that LES may be cured. Decided to make a special LES treatment equipment, stop all medical treatments, and use LES to treat the area of bedsores, the projection area of the kidneys, and the legs. After a long period of treatment, a miracle appeared. Within 3 weeks, bedsores New granulation began to grow from the inside. The exposed bones had been covered by the regrown granulation tissue. Kidney failure was also significantly improved. The ulcers on the legs began to dry out and new granulation was formed. After more than 3 months, the bedsores were completely healed, the kidney failure was normal, and most of the legs had new muscles and new skin. This extremely critical case plus some other similar cases found in the experiment show that no matter how complex and a variety of critical diseases are accompanied by one, LES from nature should be able to solve it. For patients with various diseases, such as LES natural therapy. The hope of possible recovery. The doctors felt incredible when they saw the photos before and after. The applicant also hopes to approve it as soon as possible to relieve the pain of more similar patients. See attached FIG. 8. (Photo of 4 degree bedsore.)

FIGS. 8-1 and 8-2 shows a picture of the basic healing of a 4th degree bed sore.

FIGS. 8-3 shows that the striated muscle syndrome of the legs has been treated with LES, and the ulcer has begun to dry out, and it is moving in a positive direction.

FIGS. 8-4 shows, after LES treatment, the striated muscle syndrome of the leg is basically gradually healed gradually.

FIGS. 8-5 shows that severe bedsores are close to healinge shows an old professor over 90, who miraculously survived and can walk alone.

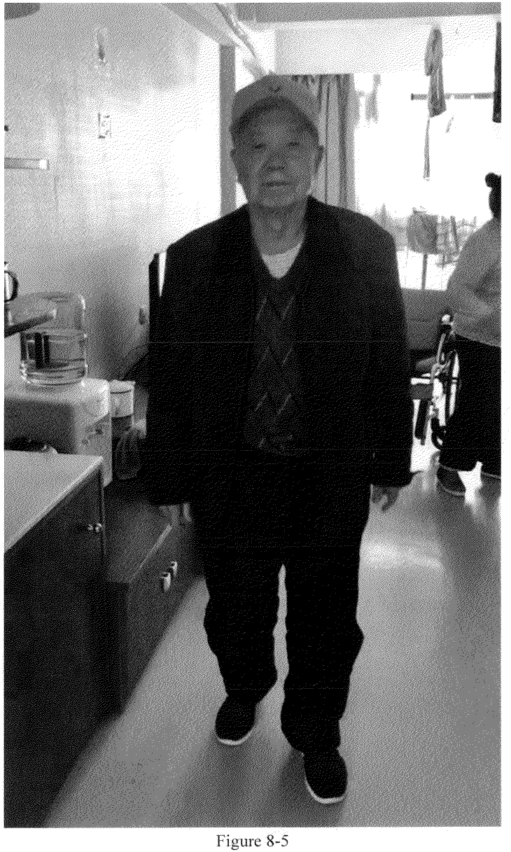

Example 5. The hospital diagnosed that an elderly woman over 80 years old had 6 blood vessel embolism in the heart, and 6 blood vessel bypass surgery was urgently needed. However, the establishment of a medical team in a hospital does not ensure the success of the operation. The old lady’s son is engaged in medical research in the United States. When he heard the news, he immediately contacted the doctor to take all life-saving measures. He also flew back to Shanghai and immediately contacted the applicant to urgently prepare a LES spectrum room for his mother. I thought that after only two weeks, the old lady felt the heavy plate on her chest disappear. Two months later, she went to the hospital for an examination and found that all 6 blood vessels were back to normal. These authoritative cardiologists asked very curiously which drugs were used. The old lady’s son said that I have been studying and understanding the trends and actual effects of various therapies around the world. I once heard that a friend once participated. In the research work of LES, it is mentioned that LES natural therapy does not require drugs; it has no side effects; it can detect and repair diseases intelligently, and has been tested in patients with cardiovascular diseases. The use of LES improves cardiovascular conditions, and it may hopefully avoid cardiovascular bypass surgery to save lives and consume a lot of medical expenses and energy. Unexpectedly, he was lucky to be successful in the experiment. This is a direction worthy of cardiovascular research and development. How many patients with cardiovascular disease are between life and death. I believe that you, like the applicant, hope that it can be used in cardiovascular clinics as soon as possible to save those precious lives. See FIG. 9, LES can significantly improve red blood cell aggregation within 10 minutes. Before using LES, the blood viscosity was higher(see the first picture in FIG. 9). After 10 minutes of use, the blood viscosity of LES will increase (see the second picture in FIG. 9). After using LES for 20 minutes (see the third picture in FIG. 9), the blood viscosity will increase significantly.

Example 6. An old man over the age of 80 suffers from cardiovascular disease in his internal organs. He is also a patient with rheumatism. Due to rheumatism, many joints of his hands and feet are severely deformed, making it difficult to walk or hold things. I went to a big hospital and saw no effect. He believes that LES has an intelligent search and repair effect on the human body. After six months of continuous use, the cardiovascular embolism disappeared, the deformities of the hands and feet caused by rheumatoids returned to normal, the hair became black and thick, and the skin of the face and body changed. It is white and tender. The LES method of delaying aging is of great significance to the United States and the world.

### Examples of BST Used in Agriculture

Example 7. Using LES to influence wheat seeds can repair and activate their internal molecular biological functions, regulate genes and proteins, increase the biological activity of seeds and the functions of stress resistance, drought resistance, diseases and insect pests, and maintain the original seed gene characteristics It is only used for intelligent regulation and repair of genetic problems, avoiding some human disputes. Compared with the control group, the yield, nutrients and taste have been significantly improved, which is of great value to global agriculture.

Example 8. LES plays an important role in watermelon cultivation. Using LES to affect watermelon seeds also plays an important role in the molecular biology of seeds. It also has intelligent repair and regulation effects on genes and proteins in watermelon seeds. The harvested watermelon is about 20% heavier than the control group, and its sweetness is also higher than that of the control group. 2-3 sweetness.

In Example 9, LES + Ag&Cu Zelile technology treats peanuts, in addition to increasing the yield, the nutrient content of peanuts is also significantly increased. In this peanut comparison experiment, the significance of increasing nutrient content is as follows: LES technology discusses the analysis results of peanut nutrient content, and is analyzed by agricultural animal nutrition experts. Conclusion: The composition of Yingqiao in the treatment group is higher than that in the control group, indicating that LES technology has an effect on the growth, metabolism and nutrient accumulation of peanuts. Storage has a promoting effect. Lysine, valine, leucine, threonine, etc. are essential oxygen acids for humans and animals. These amino acids cannot be synthesized by the human body, they are all ingested from food. Therefore, spectral processing improves the total amount of essential amino acids with nutritional significance. Minerals are also essential in the human body, and their functions include regulating body fluids to form a supporting framework and regulating chemical reactions in the body. In the processed peanuts, the content of the eight mineral elements tested increased. The role of these elements is: calcium, magnesium, etc. together with phosphorus form a skeleton system to support the body’s calcium element and participate in blood coagulation, nerve tissue regulation, and participate in the control of heart and muscle activities. Potassium: has an effect on intracellular metabolism, can regulate the absorption of cardiac smooth muscle, and affect the excitability of nerve and muscle tissue. Sodium: When it is involved in the regulation of body fluids, muscles and nerves, it enters cells and causes compensatory changes in the osmotic balance of cell membranes. Magnesium: The function of soft tissue, bone and nerve tissue must be regulated by normal calcium/mg balance. It is also the basic activator of phosphotransferase. Iron: The main role is to form hemoglobin, which is responsible for oxygen transport and cellular respiration. Copper: It is a component of tyrosinase (polyphenol oxidase) and other enzymes. It is related to iron. Adding copper to the feed obviously promotes the growth of animals. Manganese: It is a catalyst for metabolic activities, such as an activator of phosphatase and arginase. Zinc: is a component of carbon dehydratase and alkaline phosphatase. Vitamin E, also known as tocopherol, plays an important role in the reproductive system and is an antioxidant in the human body. When absorbed by the body, it enters the body together with food fat and can be stored in the body.

The applicant believes that this result is not only limited to peanuts, but will also have a positive impact on crops such as those affected by LES technology to increase yield, increase nutrients and improve taste.

### Examples of BST in Plants

Example 10: The influence of LES on wood restoration in Brazil. The applicant of the present invention has a piece of Brazilian wood in his home. The applicant found that the Brazilian wood leaves were dry from the top of the leaf to the bottom of the leaf, so he cut off the dry part with scissors. The more you cut, the drier it will be. I decided to use LES to irradiate the bottom of Brazilian wood. Unexpectedly, after a week, the dry part stopped developing, and the green leaves began to grow, eventually reaching about two floors. This is an example of restoring plants.

Example 11. The influence of LES on Clivia. Clivia is the favorite flower of many families. It usually blooms once a year, but no matter how carefully planted, many people do not bloom for many years. The applicant of the present invention patent used LES to influence two Clivia at home. He did not expect to bloom at least three times a year for many years, the flowering period is very long, the colors are colorful, and the flowers are big and full.

### The Impact of LES on Animal Husbandry

Example 12. In some places, it is difficult to obtain purebred livestock breeds without spending a lot of money. The applicant of the present invention cooperated with experts in animal husbandry and proposed to use LES to start farming from the basic primitive state of animal husbandry. The applicant and the animal husbandry experts cooperated to complete the entire process from the combination of oocytes and sperm to embryo formation, and then from cryopreservation to frostbite repair. Then, collect high-quality oocytes from the abundance of purebred cattle, place them in the simulated uterine environment of LES, and mature after 1-2 days, and then combine with sperm to form high-quality embryos. The cryogenic liquid nitrogen tank can store 5,000 embryos, and you can obtain purebred cows by being born in ordinary cows. This method can standardize the breeding of various livestock and animals, as well as some rare animals, birds and poultry.

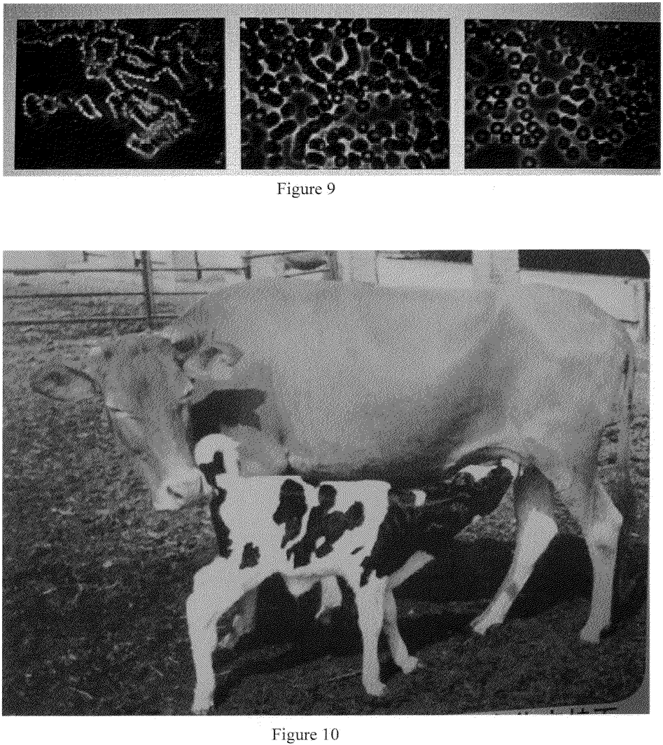

See FIG. 10, the local inferior cattle gave birth to a baby and gave birth to a purebred New Zealand cattle.

### The Effect of LES on Infertility

Example 12: Nearly 40 couples have found the applicant of the present invention, hoping to help research and solve their long-term marital infertility, and multiple artificial insemination is invalid. The applicant and doctor of the present invention use the LES method to treat the couple every day. Unexpectedly, they became pregnant within more than a month, gave birth to a healthy son, and walked and talked before the age of two.

### The Role of Longevity

The applicant cooperated with doctors to provide rehabilitation and health care services for some middle-aged and elderly people. It was found that they suffered from a variety of diseases. Even some large hospitals also found intractable diseases, but they were affected by the life energy spectrum LES, and they got significant Improved, and even restored health. Some elderly people are even comfortable and healthy beyond the age of 90, and some elderly people are fortunate to exceed the age of 100.

The above are some experimental work completed by the applicant. It has been found to prove the widespread use of LES. The applicant should be asked what the LES is for. The applicant believes that since LES is derived from nature, it should be possible to bring everything in the world into the intelligent field of LES.

Millimeter wave (mmWave) refers to electromagnetic waves with a frequency range of 30 GHz to 300 GHz. Because the wavelength of 30 GHz electromagnetic wave is 10 millimeters, the wavelength of 300 GHz electromagnetic wave is 1 millimeter. Strictly speaking, above 10 millimeters should have entered the centimeter wave. But even if some electromagnetic wavelengths enter the centimeter wave, people are still used to calling it millimeter waves or centimeter waves. But in fact, millimeter wave is just a conventional name. Some people think that electromagnetic waves with a frequency range of 20 GHz (wavelength 15 mm)-300 GHz can be regarded as millimeter waves.

For a long period of history, the millimeter wave band was a wild land. why? Because almost no electronic components or equipment can send or receive millimeter waves. There are two reasons. The first reason is that the role of millimeter waves has not been discovered at that time, there is no demand for bandwidth and data rate in the communications field, and there is no market demand for millimeter waves. And millimeter wave has some obvious limitations, such as too much propagation loss, too small coverage, etc. The second reason is that millimeter waves are too expensive. Producing sub-micron-sized integrated circuit components that can work in the millimeter wave frequency band has always been a challenge. Overcoming propagation loss and improving coverage also means a lot of money investment. However, in the past ten years, the application of millimeter waves in communications and other aspects, coupled with the development of quantum technology, because millimeter waves are also involved, have changed everything.

With the emergence of 5G communications, and the recent birth of 6G communications in China, the use of millimeter waves for communications has become very crowded. I believe it is bound to seek a breakthrough in the field of microwaves. The wide spectrum of LES is also between micrometers and millimeters. The LES, which originated in nature, is responsible for the connection with all things in nature, and the communication between the earth and the universe. Therefore, humans use this natural function of LES to establish a novel and special intelligent communication method, which will help people intelligently establish the connection between people and all things in nature, as well as the connection between the earth and the universe. become possible.

Now the United States and China attach great importance to the cooperation of industry, university and research in the field of quantum technology to promote industrial applications such as quantum communications and quantum networks. At present, the application prospect of quantum technology is not completely clear, and it will take a long time to move from “laboratory” to “industrial chain”. However, LES, which is very closely related to quantum technology, has carried out a lot of exploration in basic research and application, and has achieved meaningful and innovative results in various related fields. The applicant believes that if LES and quantum technology are combined, LES quantum science will be formed. Will likely bring a breakthrough development for quantum technology from research to application. Under the continuous exploration of the global scientific community, it is believed that the more anthropomorphic LES artificial intelligence Al system will appear soon, and it will be in the field of life and health. It is intuitive, accurate, does not require drugs, has no side effects, intelligently finds and repairs any unfavorable factors affecting health, and helps people live every day in a truly healthy and comfortable manner. The LES intelligent Al system will also play an important role in the agricultural field from molecular biology of seeds to development and growth results, so that agricultural products can carry and transmit the life energy information of the earth in terms of nutrients, yield, sense of presence, and energy. All aspects will play an important role. In the field of animal husbandry, the optimization of animal husbandry’s varieties has improved the nutrient content of meat and milk, increased production, and made the taste more delicious and tender. The balance of LES in the human health system, and the reform and progress in agriculture and animal husbandry will promote the obvious improvement and enhancement of human health. The applicant believes that: LES is a good innovative assistant for humans to solve the problem of disease. As long as you hand over the refractory diseases you encounter to LES, it will quickly search and judge through intelligence, and quickly make the refractory diseases fundamental Shift to normal. In experiments, some difficult diseases that even big hospitals and doctors find it difficult to cope with are due to the fact that pharmaceutical companies have not developed corresponding therapeutic drugs, and doctors cannot innovate no matter how capable they are. The LES has the ability to search and judge intelligently. For the same person, yesterday’s situation can be completely different from today’s situation. It is possible that the patient has not noticed it, but the LES has already accurately judged and identified it. Therefore, the applicant believes that the LES intelligent health detection system will also be developed with the efforts of scientists.

LES will work with quantum technology to form the LES quantum internet and LES anthropomorphic intelligent thinking system, which will help people liberate people from the complicated and laborious human social environment. People only need to relax while thinking about some major innovations that LES is difficult to handle. After finding the corresponding breakthrough point, the problem is handed over to LES to improve it. Such a more innovative, harmonious and happy global village may be formed.

If you have your support, it may bring a series of transformative changes to all the growth and development. I believe we can see some clues from the provisional patent. The applicant filed a provisional application for LES to the US Patent and Trademark Office, indicating that the glorious future of LES for mankind and the natural world can begin. The applicant salutes you here! The applicant will ask a patent attorney to formally assist in completing this formal patent application.

The applicant hopes that through U.S. patent protection, the impact and improvement of LES on biology are comprehensive, so as to cooperate with related industries as soon as possible, so as to affect as many people as possible in the U.S. and around the world. The approval of the patent for the present invention is indeed of great significance to the development of the United States, the world, and the development of life energy science engineering and large-scale industries.

What is claimed is:

1. A method for measuring a life energy spectrum (LES) derived from the natural world that can influence on humans and communication between humans and all things in nature, wherein the life energy spectrum is in a wide spectrum range of 0.2 micrometers to 15 millimeters.

2. The method according to claim 1, wherein the life energy spectrum is a natural micro-millimeter wave broad spectrum LES.

3. The method according to claim 1, wherein the life energy spectrum has following three characteristics:
(1) the life energy spectrum derived from nature indicates that humans research and apply natural essences from the natural world, and use the essence of nature for all aspects of human survival and life;
(2) study of living organisms makes the study of life closer to a true state of life;
(3) add artificial intelligence technology to make the life energy spectrum more intelligent and accurate in finding and repairing problems for life and everything.

4. A method for utilizing a life energy spectrum (LES) that can influence on humans and communication between humans and all things in nature, wherein the life energy spectrum can realize effective regulation from the inside of living substances.

5. The method according to claim 4, wherein an inner influence of the life energy spectrum is applied on everything comprising:
(1) carriers of nucleic acid genetic information;
(2) protein expression of life information: its synthesis constitutes a main pathway of genetic information in organisms; its biological activity shows the necessity of life energy;
(3) characterization of biological information metabolism is a core of molecular biology research;
(4) the life energy spectrum does not interfere with each other at the same time to tens of thousands of reactions in living cells of the organism, cooperates with each other, and carries out their metabolism in an orderly manner, and is intelligently suitable for the organism in terms of synthesis, decomposition speed and quantity.

6. The method according to claim 4, wherein the life energy spectrum is in a wide spectrum range of 0.2 micrometers to 15 millimeters.

7. The method according to claim 4, wherein the life energy spectrum can influence life from a molecular level, or from aspects of cells, genes, and proteins.

8. The method according to claim 4, wherein the life energy spectrum can control infection hazard of virus from the molecular level, from the aspects of cells, genes, and proteins.

9. The method according to claim 4, wherein the life energy spectrum can be used to treat diseases.

10. The method according to claim 9, wherein the life energy spectrum can be used to treat acquired immune deficiency syndrome, kidney failure, malignant tumor, striated muscle ulcers, blood vessel embolism, cardiovascular disease, or infertility.

11. The method according to claim 4, wherein the life energy spectrum can be used to influence plant seeds to repair and activate internal molecular biological functions of the plant seeds.

12. The method according to claim 4, wherein the life energy spectrum can be used in an anthropomorphic LES artificial intelligence (Al) system in a field of life and health.

13. The method according to claim 4, wherein the life energy spectrum can be added with intelligent functions.

14. The method according to claim 4, wherein the life energy spectrum can be used for an LES water functional nutrient solution, LES contraceptives, growth of rare drugs, LES nano silver fiber, fermentation tanks, various chips and the above-mentioned graphene film structure, fields of smart chemical energy, electronics, communications and services, LES sleep quilt and LES bedding series products.

15. A device for generating life energy spectrum (LES), wherein the device can generate a life energy spectrum simulating a life energy spectrum derived from the natural world.

16. The device according to claim 15, wherein the device is an analog LES generator.

17. The device according to claim 15, wherein the device is a wide-spectrum, high-power solid-state oscillator.

18. The device according to claim 15, wherein amplitude and width of the life energy spectrum can be controlled.

19. The device according to claim 15, wherein the life energy spectrum can be added with intelligent functions.
